(19) United States
(12) Patent Application Publication
(10) Pub. No.: US 2025/0229109 A1
(43) Pub. Date: Jul. 17, 2025

(54) MONITORING TISSUE PERMEABILITY DURING ULTRASOUND PROCEDURES

(71) Applicant: Insightec Ltd., Tirat Carmel (IL)

(72) Inventors: Michael PLAKSIN, Tirat Carmel (IL); Yoav LEVY, Hinanit (IL); Rafi DE PICCIOTTO, Tirat Carmel (IL); Achal Singh ACHROL, Miami, FL (US)

(21) Appl. No.: 18/849,347

(22) PCT Filed: Mar. 22, 2023
(86) PCT No.: PCT/IB2023/000168

§ 371 Date: Sep. 20, 2024

Related U.S. Application Data
(60) Provisional application No. 63/322,617, filed on Mar. 22, 2022.

(51) Int. Cl. A61N 7/00; A61B 90/00

(52) U.S. Cl. CPC A61N 7/00; A61B 90/37; A61B 2090/374; A61N 2007/0021; A61N 2007/0065; A61N 2007/0095

(57) ABSTRACT

The disclosure relates, in part, to targeted drug delivery using an ultrasound procedure and, more particularly, to systems and methods for the measurement of levels of drug delivery and certain aspects of metabolism, such as drug metabolism, in the brain tissue.

### CROSS-REFERENCE TO RELATED APPLICATIONS

This application claims priority to U.S. Provisional Application No. 63/322,617, filed on Mar. 22, 2022, the entire contents of which are incorporated herein.

### FIELD OF THE INVENTION

The invention relates generally to targeted drug delivery and, more particularly, to systems and methods for enhancing targeted drug delivery using an ultrasound procedure.

### BACKGROUND

The assessment of efficiency of drug delivery is very important for making crucial clinical decisions. Biomarkers, which provide an insight into indicators of normal or pathological biological processes, are routinely analyzed to evaluate responses to drugs and other medical interventions. Liquid biopsy is a non-invasive technique used for sampling a subject's blood for the detection and measurement of the biomarkers. The results of the analyses that follow liquid biopsy (e.g., assessment of the amounts and/or activities of proteins/enzymes, metabolites, nucleic acids, karyotypes, mutations, genetic markers, etc.) allow drawing inferences about disease progression, efficacy of drugs or other intervention, stability and metabolism of drugs, etc. Consequently, liquid biopsy is very helpful for diagnosis, selection of therapy, determination of doses and dose regimens, efficacy, etc. The use of liquid biopsy has made strides for diverse diseases including genetic disorders, cancer, infectious diseases, metabolic diseases, environmental diseases, allergies, autoimmune disorders, etc. However, its use in diseases of certain tissues is limited because of the lack of permeability of the biomarkers from those tissues to blood. For example, the blood-brain barrier (“BBB”) prevents the transit of biomarkers from the central nervous system (CNS) to blood. Therefore, the diagnosis and treatment of diseases such as stroke, multiple sclerosis (MS), neurodegenerative disorders, Alzheimer's disease (AD), Parkinson's disease, epilepsy, and traumatic brain injuries is challenging without the use of more complicated tests. Other such barriers that hinder the transfer of biomarkers from tissues to blood include the blood-testis barrier, blood-retina barrier, blood-placenta barrier and blood-thymus barrier.

Drug-delivery systems are frequently developed with the objectives of lowering the overall administered therapeutic dose of a pharmaceutical, increasing its residence time, prolonging its release over time, and enhancing the targeting of diseased tissues. The above barriers impede targeted delivery of drugs. One conventional approach used for enhancing targeted drug delivery involves the use of ultrasound. For example, exposing target tissue to ultrasound may increase its permeability, allowing a higher proportion of a drug dose to exert a therapeutic effect on the target region. In another approach, microbubbles are injected to the blood stream to enhance vascular permeability to various drugs via ultrasound and biomechanical interaction with the microbubbles. Moreover, drugs can be encapsulated in “nanoparticles” and injected with microbubbles at the target region; application of ultrasound will cause the microbubbles to undergo structural mechanical deformations and/or temperature increase, thereby triggering release of the encapsulated drug. Similarly, ultrasound energy may be applied to chemically activate drugs administered into the target region.

In particular, numerous studies have explored the safety and the efficacy of using ultrasound to open the blood-brain barrier (BBB) to permit passage of neurological therapeutics. In general, the BBB impedes passage from blood to brain parenchyma of molecules with molecular weight higher than about 400 Daltons, causing the vast majority of neuro-therapeutic agents and biomarkers to be ineffective. Ultrasound has shown safe and effective BBB opening mediated by oscillation of intravascular microbubbles. Advantages to its use include its non-invasive character, its millimeter-scale special precision, and the reversibility of its effects on the BBB.

Ultrasound in combination with liquid biopsy has made investigation of some central nervous system biomarkers feasible. However, even with the altered BBB, there is substantial heterogeneity in permeability that prevents reliable detection and/or measurement of biomarkers. For instance, the quantities of the biomarkers in two measurements in the same subject are also not comparable to each other. This hinders the efforts to monitor disease progression, reliably deliver exact amounts of drugs and monitor the efficacy of clinical interventions. Therefore, there remains a need to be able to predict the extent of opening of a barrier by ultrasound (or analogous therapy), the extent of delivery of drugs, monitor changes in biomarkers, and compare one application of opening of a barrier by ultrasound (or analogous therapy) with another application. Without accurate predictions, it is difficult to make critical decisions regarding therapy.

### SUMMARY

The present invention provides systems and methods for assessing tissue permeability enhancements in a target region using an ultrasound procedure. For example, the target tissue may normally be permeable to molecules having a size less than 400 Daltons, but the therapeutic agent may have a size of 1,000 Daltons or more and therefore is blocked from entering the target region. Upon application of ultrasound, tissue in or regulating access to the target region may be disrupted, and consequently, the permeability thereof may be increased. The permeability level and/or the size of the tissue region in which the permeability has been increased may depend on the intensity and/or duration of the ultrasound application. Accordingly, by adjusting ultrasound parameters, the tissue permeability of the target region can be increased to a desired degree to allow the therapeutic agent to penetrate and/or diffuse therein. The present disclosure also provides systems and methods for normalization of degree of permeability between instances of sonication to allow the delivery of precise amount of drug to target tissue every time.

Several techniques are herein presented for monitoring the permeability of target tissue during or following exposure to ultrasound. In general, they involve the use of molecules or molecular complexes having a size range similar to that of the therapeutic of interest, and whose passage through the disrupted target tissue can be observed and quantified.

In a first approach, the relaxation rate ΔR₂*, a physical parameter that is regularly measured in MRI systems as a safety measure, is used to measure and predict the extent of BBB opening during treatment. ΔR₂* can be used to predict liposome concentration, for example. The liposomes may be conjugated to an MRI-visible species such as Gd ions (see, e.g., “Nonlinear ΔR₂* Effects in Perfusion Quantification Using Bolus-Tracking MRI,” Mag. Res. In Medicine, 61:486-492 (2009), which is incorporated herein by reference) but such contrast agents are not essential to this approach. Alternatively, the transverse relaxation time T₂* may be used instead of or in addition to ΔR₂*. Once again, contrast agent is unnecessary.

Another MRI parameter that may be used to assess tissue permeability is the longitudinal relation time T₁, which is a measure of the time taken for spinning protons to realign with the external magnetic field during an MRI procedure. T₁ may be used in conjunction with ΔR₂* in estimating a tissue permeability level or may be employed independently as a measure of permeability (although the T₁ measurement may be obtained only once in a given treatment session).

In another approach, BBB permeability to different molecules is predicted based on the entrance of similarly sized nanodroplets or liposomes filled with one or more acoustic agents. For example, sonodynamic treatment is a non-thermal ultrasonic technique using sonochemical effect of cavitation bubbles. Rose bengal (RB) is sonochemically active and reduces the cavitation threshold, and therefore can support sonodynamic treatment. Other sonodynamic agents are include titanium (Ti)-based nanomaterials (particularly TiO₂) titanium hydrides, titanium carbides, Xanthene dyes (such as fluorescein, tetrachlorofluorescein, eosin Y, erythrosine B, and phloxin B), protoporphyrin IX, hematoporphyrin monomethyl ether (HMME), chlorin e6 (Ce6), and silicon-based nanoparticles. Encapsulation of these agents in liposomes can be achieved by reverse-phase evaporation and extrusion. An alternative is to use acoustically responsive phase shift nanodroplets. The size of these agents and other detection agents (without limitation, e.g., an magnetic resonance imaging (MRI)-visible contrast agent comprising ionic gadolinium (Gd)) may be adjusted by complexing (e.g., by chemical conjugation) to entities such as a polymer (without limitation, e.g., polyethylene glycol and dextran) or encapsulation in a liposome. Accordingly, these agents and other detection agents may be used for normalization of therapeutic agents in desired molecular weight range.

In yet another approach, BBB permeability to different molecules is predicted based on the exit of similarly sized biomarkers from target tissue into the bloodstream. Non-limiting examples of target tissue-specific biomarkers include the central nervous tissue-specific markers such as the neuron-specific enolase (NSE) and S100 calcium binding protein B (S100B), which are not usually found in blood.

Accordingly, in a first aspect, a system is provided for disrupting target tissue for treatment. The system includes an ultrasound transducer for sonicating the target volume to cause disruption of the target tissue therein and thereby increase a permeability thereof. The system also includes a controller configured to (i) monitor a degree of permeability of the target tissue caused by the disruption and (ii) cause the ultrasound transducer to cease sonicating the target volume when the degree of permeability reaches a threshold.

In some embodiments, the degree of permeability is calculated based on the measurement of a molecule that is present (and in some cases, predominantly present) in the target tissue but substantially not present in tissues outside the target tissue under normal conditions. By “substantially not present” is meant that the background level of the molecule is sufficiently small that its increased presence due to barrier permeability may be readily detected. In some embodiments, the target tissue is brain. In some embodiments, the molecule that is predominantly present in the target tissue but substantially not present in tissues outside the target tissue under normal conditions. Illustrative, non-limiting examples of molecule that is present in the target tissue but not in tissues outside the target tissue include NSE and S100B. In some embodiments, the degree of permeability is calculated based on the measurement inside the target tissue of a detection marker that is administered a subject in which the degree of permeability is calculated. In some embodiments, the detection marker and/or the is complexed with a polymer, a liposome, a quantum dot, a dextran, or the like. In some embodiments, the detection marker is a MRI-visible contrast agent, which optionally comprises ionic Gd.

In some embodiments, the degree of permeability corresponds to an upper limit of the size distribution of molecules or molecular complexes passing through the disrupted target tissue. The degree of permeability may alternatively or in addition correspond to a concentration of molecules or molecular complexes that have passed through the disrupted target tissue. In some embodiments, the degree of permeability is calculated based on the measurement of a molecule that is in the size range, and present in the target tissue but not in tissues outside the target tissue.

In some embodiments, the degree of permeability is compared among at least two applications of sonication on different days administered to the same subject. In some embodiments, the degree of permeability is compared among at least two applications of sonication administered to different subjects.

In some embodiments, the degree of permeability is normalized using the measurement of the detection agent or the molecule that is predominantly present in the target tissue but substantially not present in tissues outside the target tissue under normal conditions. In some embodiments, the target tissue is brain, and the molecule that is predominantly present in the target tissue but substantially not present in tissues outside the target tissue under normal conditions. Illustrative, non-limiting examples of molecules that are predominantly present in the target tissue but substantially not present in tissues outside the target tissue include, but are not limited to, the brain proteins NSE, S100B, astrocytic glial fibrillary actin protein (GFAP) and the neuronal ubiquitin carboxyl-terminal hydrolase isoenzyme L1 (UCH-L1), myelin basic protein (MBP), tau, and neurofilament light chain (NfL). In some embodiments, the species of NSE that is detected has a molecular weight of about 78 kDa. In some embodiments, the species of S100B that is detected has a molecular weight of about 22 kDa. In some embodiments, the measurement of the species of NSE and/or S100B is used for normalization of transit of a therapeutic agent in the molecular weight range of about 60 to about 100 kDa, and/or about 15 to about 30 kDa, respectively.

In some embodiments, the detection marker is conjugated with a polymer (e.g., a dextran or a polyethylene glycol) that is in a defined molecular weight range corresponding to the molecular weight range of a therapeutic agent. In some embodiments, the detection marker is complexed a liposome having a defined size range corresponding to the size range of a therapeutic agent.

The size range of such molecules or molecular complexes may correspond to a monoclonal antibody, a viral vector, a liposome, a nucleic acid (e.g., miRNA, mRNA, etc.), or a protein. Some embodiments use markers with relevant size. Some embodiments select markers with a size comparable to a desired drug (e.g., a monoclonal antibody, a viral vector, a liposome, a protein, and/or a nucleic acid). Molecules or complexes can be similarly sized to a drug of interest but with minimal therapeutic effect when administered.

In some embodiments, the system further includes a MRI device. The controller may be responsive to the MRI device may evaluate the degree of permeability based on a measurement of at least one of ΔR₂*, T₁ or T₂*. A relationship between the measurement and the degree of permeability may be established by calibration, which may include, in some embodiments, a machine learning component. Machine learning may alternatively or in addition be used to improve calibration curves with time. The controller may be configured to evaluate the degree of permeability without a contrast agent.

In some embodiments, the target species corresponds to a molecule or molecular complex coupled to a visualization agent. The visualization agent may be a contrast agent or a fluorophore, e.g., ionic Gd. The molecule or molecular complex may be a liposome, a quantum dot, or a dextran.

In some embodiments, the transducer is configured also to detect acoustic signals. This is not acoustic response during treatment sonication, but post sonication diagnostic. The controller may be responsive to acoustic signals detected by the transducer and may evaluate the degree of permeability based on an acoustic measurement of a target species. The target species may correspond to a molecule or molecular complex coupled to an acoustic agent. The controller may be further configured to evaluate the degree of permeability at least in part based on a combination of an acoustic measurement and an MRI image. The controller may update a target acoustic dose level at least in part based on a combination of an acoustic measurement and an MRI image.

In various embodiments, the controller is configured to control an ultrasound treatment based at least in part on the updated target acoustic dose level. The treatment may include selecting initial desired acoustic dose, sonicating using the desired acoustic dose and measure the acoustic dose, computing MRI (e.g., Rz) maps, updating the desired acoustic dose based on MRI, and controlling the next sonication based on the revised acoustic dose. Some embodiments update the acoustic dose during a session, after each sonication, and once the controller has established what works for a particular patient, it may use that acoustic dose in sessions going forward. Different values (of ΔR₂* and desired acoustic dose) may be used for different tissue types. Once updated, the target acoustic dose level may be used repetitively during treatment or even during the next treatment. This may cover the situation in which a patient gets a treatment in the MRI and goes home, and may alleviate the need for MRI guidance during the next session. The parameters of subsequent treatment sessions may be changed based on parameters of the prior treatment and observed clinical results (e.g., in case of insufficient response, increase the dose).

The degree of permeability may correspond to a concentration of agent-coupled molecules or molecular complexes having a target size range and which have passed through the disrupted target tissue. The visualization agent may be a contrast agent and the concentration may be determined at least in part by either a wash-in or a wash-out time. The wash-out time may correspond to a rise or a decline in an acoustic signal characteristic with respect to a threshold. The acoustic agent may be a contrast agent and the signal characteristic may be the reflected amplitude of an acoustic signal emitted by the transducer.

In another aspect, a method is provided for disrupting target tissue for treatment. In various embodiments, the method includes sonicating the target volume to cause disruption of the target tissue therein and thereby increase a permeability thereof; monitoring a degree of permeability of the target tissue caused by the disruption; and ceasing sonication of the target volume when the determined permeability reaches a threshold.

In some embodiments, the degree of permeability is calculated based on: (i) a measurement of a level of a molecule that is predominantly present in the target tissue but substantially not present in tissues outside the target tissue under normal conditions in a biological sample from the subject in which the degree of permeability is being monitored, and/or (ii) a measurement of a detection marker inside the target tissue, wherein the detection marker has been administered to a subject in which the degree of permeability is being monitored.

In some embodiments, the target tissue is brain. In some embodiments, the biological sample is blood. In some embodiments, the molecule that is predominantly present in the target tissue but substantially not present in tissues outside the target tissue under normal conditions. Illustrative, non-limiting examples of molecules that are predominantly present in the target tissue but substantially not present in tissues outside the target tissue include, but are not limited to, the brain proteins NSE, S100B, GFAP and UCH-L1, MBP, tau, and NfL. In some embodiments, the species of NSE that is detected has a molecular weight of about 78 kDa. In some embodiments, the species of S100B that is detected has a molecular weight of about 22 kDa. In some embodiments, the measurement of the species of NSE and/or S100B is used for normalization of transit of a therapeutic agent in the molecular weight range of about 60 to about 100 kDa, and/or about 15 to about 30 kDa, respectively.

In some embodiments, the detection marker is conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is a MRI-visible contrast agent, which optionally comprises ionic Gd. In some embodiments, sonication comprises at least two applications on different days in the same patient or different applications in different patients.

In another aspect, a method is provided for comparing a degree of permeability of a target tissue in a subject among at least two applications of focused ultrasound treatment on different days, the method comprising: (i) providing a biological sample from the subject that received a first application of sonication to a target volume to cause disruption of the target tissue therein and thereby increase a permeability thereof, (ii) measuring the amount in the biological sample of a molecule that is predominantly present in the target tissue but substantially not present in tissues outside the target tissue under normal conditions, (iii) monitoring a degree of permeability of the target tissue caused by the disruption; (iv) providing a second biological sample from the subject that received a second application of sonication on a different day to a target volume to cause a second disruption of the target tissue therein and thereby increase a second permeability thereof, (v) measuring in the second biological sample the amount of the molecule that is predominantly present in the target tissue but substantially not present in tissues outside the target tissue under normal conditions, (vi) monitoring a degree of permeability of the target tissue caused by the second disruption; and (vii) comparing a degree of permeability of the target tissue in a subject among at least two applications of sonication on different days.

In some embodiments, the target tissue is brain and/or the biological sample is blood. In some embodiments, the molecule that is predominantly present in the target tissue but substantially not present in tissues outside the target tissue under normal conditions is a brain protein. In some embodiments, the brain protein is not usually found outside a cranial tissue (e.g., in the bloodstream). In some embodiments, the brain protein leaks outside a cranial tissue (e.g., in the bloodstream) when the blood-brain barrier is opened sufficiently to allow the leakage of molecules having size or molecular weight of the brain protein. In some embodiments, the brain protein becomes detectable in a biological sample (e.g. a blood sample) when the blood-brain barrier is opened sufficiently to allow the leakage of molecules having size or molecular weight of the brain protein. Illustrative, non-limiting examples of molecules that are predominantly present in the target tissue but substantially not present in tissues outside the target tissue include, but are not limited to, the brain proteins NSE, S100, GFAP and UCH-L1, MBP, tau, and NfL. In some embodiments, the species of NSE that is detected has a molecular weight of about 78 kDa. In some embodiments, the species of S100B that is detected has a molecular weight of about 22 kDa. In some embodiments, the species of GFAP that is detected is a monomer (50 kDa), a dimer (100 kDa) and/or a tetramer (200 kDa). Tang et al., J Biol Chem. Oligomers of Mutant Glial Fibrillary Acidic Protein (GFAP) Inhibit the Proteasome System in Alexander Disease Astrocytes, and the Small Heat Shock Protein αB-Crystallin Reverses the Inhibition, 2010; 285(14): 10527-10537. Likewise, in some embodiments, the species of UCH-L1, MBP, tau, and NfL have molecular weights of about 25 kDa, about 18,500 Daltons, about 62 kDa, and about 68 kDa, respectively. Accordingly, in some embodiments, the measurement of the species of NSE is used for normalization of transit of a therapeutic agent in the molecular weight range of about 60 to about 100 kDa. In some embodiments, the measurement of the species of S100B is used for normalization of transit of a therapeutic agent in the molecular weight range of about 15 to about 30 kDa. In some embodiments, the measurement of a species of GFAP is used for normalization of transit of a therapeutic agent in the molecular weight range of about 40 to about 250 kDa, depending on the species detected. In some embodiments, the measurement of a species of UCH-L1 is used for normalization of transit of a therapeutic agent in the molecular weight range of about 20 to about 30 kDa. In some embodiments, the measurement of a species of the MBP is used for normalization of transit of a therapeutic agent in the molecular weight range of about 15 to about 20 kDa. In some embodiments, the measurement of a species of tau is used for normalization of transit of a therapeutic agent in the molecular weight range of about 50 to about 70 kDa. In some embodiments, the measurement of a species of NfL is used for normalization of transit of a therapeutic agent in the molecular weight range of about 60 to about 80 kDa. In various embodiments, the measurement of the species of one or more of the molecules that are predominantly present in the target tissue but substantially not present in tissues outside the target tissue is used for normalization of transit of a therapeutic agent in the molecular weight range of about 15 to about 250 kDa.

In another aspect, a method is provided for comparing a degree of permeability of a target tissue in a subject among at least two applications of focused ultrasound treatment on different days, the method comprising: (i) providing a subject that received a first application of sonication to a target volume to cause disruption of the target tissue therein and thereby increase a permeability thereof, wherein the subject has received a detection marker, optionally on the same day, (ii) measuring the level of the detection marker in the target tissue, and thereby monitoring a degree of permeability of the target tissue caused by the disruption; (iii) providing a second biological sample from the subject that received a second application of sonication on a different day to a target volume to cause a second disruption of the target tissue therein and thereby increase a second permeability thereof, optionally wherein the subject has received a detection marker on the same day, (iv) measuring the level of the detection marker in the target tissue, and thereby monitoring a degree of permeability of the target tissue caused by the second disruption, and thereby monitoring a degree of permeability of the target tissue caused by the second disruption; and (v) comparing a degree of permeability of the target tissue in a subject among at least two applications of sonication on different days.

In some embodiments, the target tissue is brain. In some embodiments, the target tissue is brain and the detection marker is conjugated with a polymer (e.g., a dextran or a polyethylene glycol) that is in a defined molecular weight range corresponding to the molecular weight range of a therapeutic agent. In some embodiments, the target tissue is brain and the detection marker is complexed a liposome having a defined size range corresponding to the size range of a therapeutic agent. In some embodiments, the detection marker is a MRI-visible contrast agent. In some embodiments, the MRI-visible contrast agent comprises ionic Gd. In some embodiments, the detection marker is a positron emission tomography (PET) reporter. In some embodiments, the PET reporter is selected from 18F-florbetapir, 18F-Florbetaben, 18F-Flortaucipir, 18F-Flutemetamol and 18F-Flourodopa (F-Dopa). In some embodiments, the detection marker is a single photon emission computed tomography (SPECT) reporter. In some embodiments, the SPECT reporter is selected from 99mTc-ECD (ethylcysteinate-dimer), 123I-Ioflupane, and ^{99m}Tc-TRODAT-1).

In yet another aspect, the invention relates to a method of treating a neurological disease or disorder in a subject in need thereof, wherein the neurological disease or disorder is characterized by having a locus of abnormal production, aggregation, and/or deposition of a protein or another biomolecule in the brain, wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control.

In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some embodiments, the neurological disease or disorder is selected from the Alzheimer's Disease (AD), Parkinson's Disease (PD), Huntington's Disease (HD), amyotrophic lateral sclerosis (ALS), dementia with Lewy bodies, spinocerebellar ataxia, and amyotrophic lateral sclerosis, frontotemporal diseases, multiple system atrophy, four-repeat tauopathy and prion diseases. In some embodiments, the locus is selected from senile plaques, neurofibrillary tangles, neuronal inclusions, Lewy bodies, glial inclusions, cytoplasmic inclusions, and polyglutamine aggregates. In some embodiments, the protein showing abnormal production, aggregation, and/or deposition is selected from amyloid-β (Aβ), Tau protein, of TDP-43, α-Synuclein, FUS/TLS, SOD1, and Huntingtin.

In some embodiments, the therapeutic agent comprises a small molecule or a biologic drug. In some embodiments, the therapeutic agent is or comprises a biologic drug. In some embodiments, the therapeutic agent is selected from a gene therapy agent, an enzyme for enzyme replacement therapy (e.g., ceramidase, acid alpha-glucosidase (EC 3.2.1.20), agalsidase beta, alpha-l-iduronidase, acid sphingomyelinase, imiglucerase, glucosylceramide (GlcCer) synthase, and arylsulfatase A (ARSA); the enzyme being replaced may be administered as a purified protein, a nucleic acid encoding the active enzyme and/or as a nucleic acid that corrects defects such as modulates correct splicing), a vaccine, an antisense oligonucleotide (ASO), a protein therapeutic, a modified mRNA agent, and a RNAi agent. In some embodiments, the therapeutic agent is or comprises an antibody, antibody-like molecule or an antigen-binding fragment thereof. In some embodiments, the therapeutic agent specifically binds the protein or another biomolecule that that exhibits abnormal production, aggregation, and/or deposition. In some embodiments, the therapeutic agent is selected from a nonspecific clearing antibody (e.g., intravenous immunoglobulin aka IVIg), an anti-amyloid-β antibody (e.g., aducanumab, gantenerumab, lecanemab, and donanemab), an anti-tau antibody (e.g., semorinemab, gosuranemab, tilavonemab, and zagotenemab), an anti-TREM2 antibody (e.g., AL002), an anti-alpha-synuclein antibody (e.g., Cinpanemab, Prasinezumab, Lu AF82422, ABBV-0805, and MEDI1331), and or a combination thereof.

In some embodiments, the therapeutic agent is or comprises a small molecule drug. In some embodiments, the therapeutic agent provides one or more of synaptic plasticity, neuroprotection, reduction of inflammation, neurotransmitter receptor modulation, reduction of oxidative stress. In some embodiments, the therapeutic agent is selected from donepezil, galantamine, rivastigmine, memantine, suvorexant, carbidopa-levodopa, selegiline, rasagiline, safinamide, entacapone, benztropine, tolcapone, opicapone, nuplazid, istradefylline and amantadine, and a combination thereof.

In some embodiments, the therapeutic agent is formulated in a liposome. In some embodiments, the therapeutic agent is delivered via a viral vector.

In yet another aspect, the invention relates to a method of treating a neurological disease or disorder in a subject in need thereof, wherein the neurological disease or disorder is characterized by having a locus of abnormal production, aggregation, and/or deposition of a protein or another biomolecule in the brain, the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range.

In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the neurological disease or disorder is selected from the Alzheimer's Disease (AD), Parkinson's Disease (PD), Huntington's Disease (HD), amyotrophic lateral sclerosis (ALS), dementia with Lewy bodies, spinocerebellar ataxia, and amyotrophic lateral sclerosis, frontotemporal diseases, multiple system atrophy, four-repeat tauopathy and prion diseases. In some embodiments, the locus is selected from senile plaques, neurofibrillary tangles, neuronal inclusions, Lewy bodies, glial inclusions, cytoplasmic inclusions, and polyglutamine aggregates. In some embodiments, the protein showing abnormal production, aggregation, and/or deposition is selected from amyloid-β (Aβ), Tau protein, of TDP-43, α-Synuclein, FUS/TLS, SOD1, and Huntingtin.

In some embodiments, the therapeutic agent comprises a small molecule or a biologic drug. In some embodiments, the therapeutic agent is or comprises a biologic drug. In some embodiments, the therapeutic agent is selected from a gene therapy agent, an enzyme for enzyme replacement therapy (e.g., ceramidase, acid alpha-glucosidase (EC 3.2.1.20), agalsidase beta, alpha-1-iduronidase, acid sphingomyelinase, imiglucerase, glucosylceramide (GlcCer) synthase, and arylsulfatase A (ARSA); the enzyme being replaced may be administered as a purified protein, a nucleic acid encoding the active enzyme and/or as a nucleic acid that corrects defects such as modulates correct splicing), a vaccine, an antisense oligonucleotide (ASO), a protein therapeutic, a modified mRNA agent, and a RNAi agent. In some embodiments, the therapeutic agent is or comprises an antibody, antibody-like molecule or an antigen-binding fragment thereof. In some embodiments, the therapeutic agent specifically binds the protein or another biomolecule that that exhibits abnormal production, aggregation, and/or deposition. In some embodiments, the therapeutic agent is selected from a nonspecific clearing antibody (e.g., intravenous immunoglobulin aka IVIg), an anti-amyloid-β antibody (e.g., aducanumab, gantenerumab, lecanemab, and donanemab), an anti-tau antibody (e.g., semorinemab, gosuranemab, tilavonemab, and zagotenemab), an anti-TREM2 antibody (e.g., AL002), an anti-alpha-synuclein antibody (e.g., Cinpanemab, Prasinezumab, Lu AF82422, ABBV-0805, and MEDI1331), and or a combination thereof.

In some embodiments, the therapeutic agent is or comprises a small molecule drug. In some embodiments, the therapeutic agent provides one or more of synaptic plasticity, neuroprotection, reduction of inflammation, neurotransmitter receptor modulation, reduction of oxidative stress. In some embodiments, the therapeutic agent is selected from donepezil, galantamine, rivastigmine, memantine, suvorexant, carbidopa-levodopa, selegiline, rasagiline, safinamide, entacapone, benztropine, tolcapone, opicapone, nuplazid, istradefylline and amantadine, and a combination thereof.

In some embodiments, the therapeutic agent is formulated in a liposome. In some embodiments, the therapeutic agent is delivered via a viral vector.

As used herein, the term “substantially” means □10% by a tissue volume, and in some embodiments, □5% by a tissue volume. “Clinically significant” means having an undesired (and sometimes the lack of a desired) effect on tissue that is considered significant by clinicians, e.g., triggering the onset of damage thereto. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1A schematically depicts an exemplary ultrasound system in accordance with various embodiments of the current invention.

FIG. 1B schematically depicts an exemplary MRI system in accordance with various embodiments of the current invention.

FIG. 2 depicts one or more focal zones of ultrasound waves/pulses generated in a target volume in accordance with various embodiments.

FIG. 3 depicts presence of microbubbles in a target tissue region in accordance with various embodiments.

FIG. 4 is a flow diagram of a representative process for relating an MR parameter to trans-BBB liposome penetration in accordance with various embodiments.

FIG. 5A and FIG. 5B show relationships between ΔR₂* and Gd-liposomal concentration in accordance with various embodiments.

FIG. 6A shows a graph plot of liposome particle distribution calculated from dynamic light scattering (DLS) data in accordance with some embodiments.

FIG. 6B graphically depicts the observed relationship between R₁ and the concentration of a Gd-liposome complex in accordance with some embodiments.

FIGS. 7A-7C show a ΔR₂* map and its relationship to the locations of sub-spots in accordance with some embodiments.

FIG. 8A depicts an R₁ map indicating the presence of Gd in 100 nm liposomes and a graphical plot of R₁ against Gd concentration in rat blood; and FIG. 8B shows similar results for 50 nm liposomes.

### DETAILED DESCRIPTION

We first describe systems and methods for disrupting the BBB to permit transit of therapeutics, and then set forth techniques for assessing and monitoring the induced tissue permeability. The present disclosure is based, in part, on the discovery of techniques for assessing and monitoring the induced tissue permeability across tissues such as the BBB.

The techniques disclosed herein enable the prediction of the extent of opening of a barrier by ultrasound (or analogous therapy) and the extent of delivery of drugs. The techniques disclosed herein further allow monitoring changes in biomarkers, and comparing one application of opening of a barrier by ultrasound (or analogous therapy) with another application. With such predictions, the techniques disclosed herein assist in making critical decisions regarding, inter alia, the selection or change of therapy, developing doses and dosing regimens, monitoring efficiency of local drug delivery, and monitoring the efficacy of the treatment.

In some aspects, the techniques rely on the measurement of transit to the target tissue of a detection marker that is administered to a patient (without limitation, e.g., a MRI-visible contrast agent). In these embodiments, the detection marker transits to the target tissue after induction of permeability (without limitation, e.g., using ultrasound). In some embodiments, the molecular weight or the size of the detection marker is used for calculating the extent of transit of therapeutic agents of having molecular weights or the sizes similar to that of the detection marker. In some embodiments, the molecular weight or the size of the detection markers is used for normalization for the purpose of comparing the extent of transit of therapeutic agents during different applications of induction of permeability.

In some aspects, the techniques rely on the detection and measurement of a target tissue-specific molecule outside the target tissue after induction of permeability (without limitation, e.g., using ultrasound). In these embodiments, the molecular weight or the size of the target tissue-specific molecule is used for calculating the extent of the transit of therapeutic agents of having molecular weights or the sizes similar to that of the target tissue-specific molecule. In these embodiments, the molecular weight or the size of the target tissue-specific molecule is used for normalization for the purpose of comparing the extent of transit of therapeutic agents during different applications of induction of permeability.

In some embodiments, the degree of permeability is calculated based on normalization based on the measurement of detection markers or biomarkers disclosed herein. In some embodiments, the degree of permeability is calculated and compared between two or more different applications of ultrasound application in the same patient. In some embodiments, the degree of permeability is calculated and compared between different patients upon ultrasound application based on normalization.

1. Increasing Tissue Permeability with Ultrasound

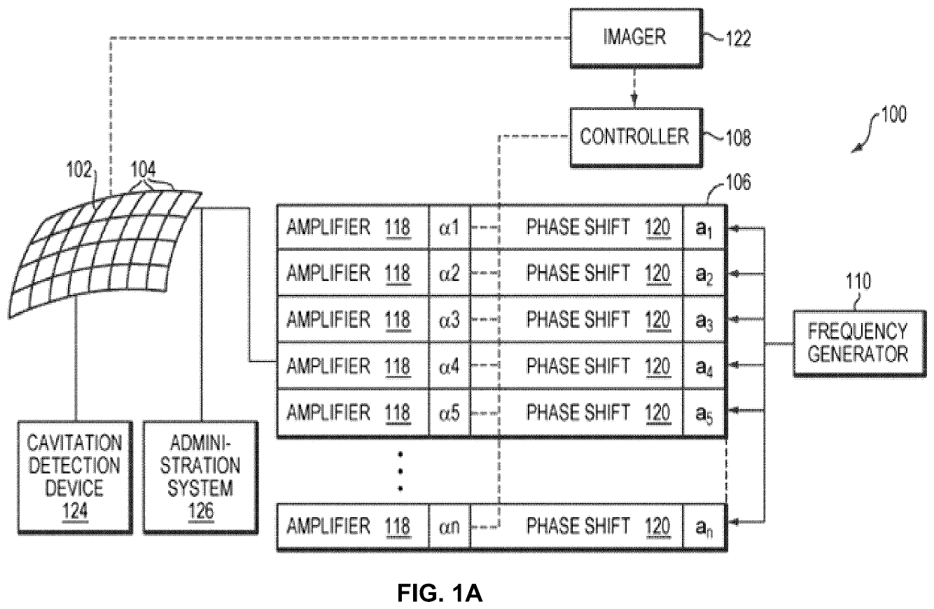

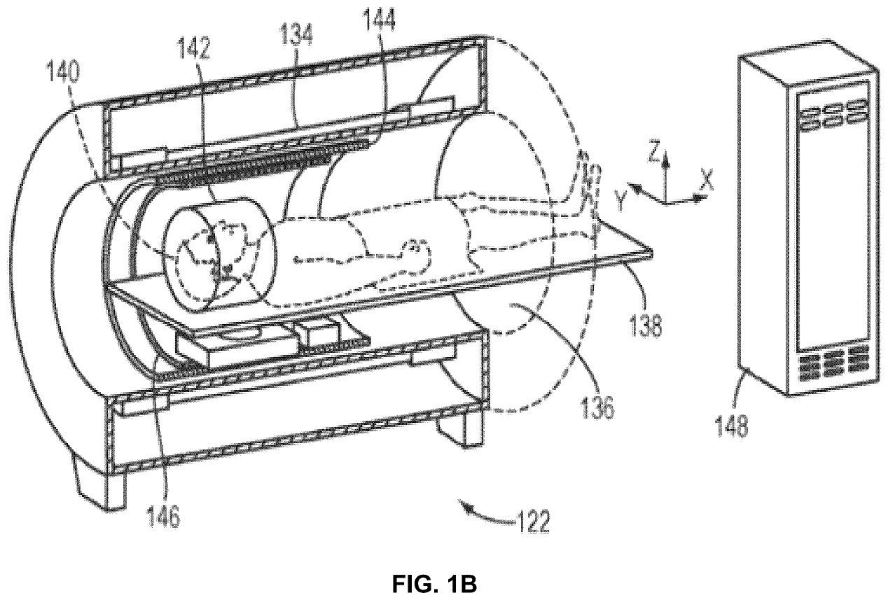

FIG. 1A illustrates an exemplary ultrasound system 100 for generating and delivering a focused acoustic energy beam to a target region for disrupting the tissue and thereby causing the tissue permeability to increase therein. In various embodiments, the system 100 includes a phased array 102 of transducer elements 104, a beamformer 106 driving the phased array 102, a controller 108 in communication with the beamformer 106, and a frequency generator 110 providing an input electronic signal to the beamformer 106.

The array 102 may have a curved (e.g., spherical or parabolic) shape suitable for placing it on the surface of the patient's body, or may include one or more planar or otherwise shaped sections. Its dimensions may vary between millimeters and tens of centimeters. The transducer elements 104 of the array 102 may be piezoelectric ceramic elements, and may be mounted in silicone rubber or any other material suitable for damping the mechanical coupling between the elements 104. Piezo-composite materials, or generally any materials capable of converting electrical energy to acoustic energy, may also be used. To assure maximum power transfer to the transducer elements 104, the elements 104 may be configured for electrical resonance at 50Ω, matching input connector impedance.

The transducer array 102 is coupled to the beamformer 106, which drives the individual transducer elements 104 so that they collectively produce a focused ultrasonic beam or field. For n transducer elements, the beamformer 106 may contain n driver circuits, each including or consisting of an amplifier 118 and a phase delay circuit 120; each drive circuit drives one of the transducer elements 104. The beamformer 106 receives a radiofrequency (RF) input signal, typically in the range from 0.1 MHz to 10 MHz, from the frequency generator 110, which may, for example, be a Model DS345 generator available from Stanford Research Systems. The input signal may be split into n channels for the n amplifiers 118 and delay circuits 120 of the beamformer 106. In some embodiments, the frequency generator 110 is integrated with the beamformer 106. The radiofrequency generator 110 and the beamformer 106 are configured to drive the individual transducer elements 104 of the transducer array 102 at the same frequency, but at different phases and/or different amplitudes.

The amplification or attenuation factors α1-αn and the phase shifts a1-an imposed by the beamformer 106 serve to transmit and focus ultrasonic energy onto the target region, and account for wave distortions induced in the tissue located between the transducer elements 104 and the target region. The amplification factors and phase shifts are computed using the controller 108, which may provide the computational functions through software, hardware, firmware, hardwiring, or any combination thereof. For example, the controller 108 may utilize a general-purpose or special-purpose digital data processor programmed with software in a conventional manner, and without undue experimentation, in order to determine the phase shifts and amplification factors necessary to obtain a desired focus or any other desired spatial field patterns at the target region. In certain embodiments, the computation is based on detailed information about the characteristics (e.g., structure, thickness, density, etc.) of the tissue located between the transducer element 104 and their effects on propagation of acoustic energy. Such information may be obtained from an imager 122. The imager 122 may be, for example, a MRI device, a computer tomography (CT) device, a positron emission tomography (PET) device, a single-photon emission computed tomography (SPECT) device, or an ultrasonography device. Image acquisition may be three-dimensional (3D) or, alternatively, the imager 122 may provide a set of two-dimensional (2D) images suitable for reconstructing a three-dimensional image of the target region and/or its surrounding region. In addition, the ultrasound system 100 and/or imager 122 may be utilized to detect the presence, type, and/or location associated with microbubble cavitation as further described below. Additionally or alternatively, the system may include a cavitation detection device (such as a hydrophone or suitable alternative) 124 to detect information associated with microbubble cavitation and an administration system 126 for parenterally introducing a therapeutic agent and/or microbubbles into the patient's body as further described below. The imager 122, the cavitation detection device 124, and/or the administration system 126 may be operated using the same controller 108 that facilitates the transducer operation; alternatively, they may be separately controlled by one or more separate controllers intercommunicating with one another.

FIG. 1B illustrates an exemplary imager namely, an MRI apparatus 122. The apparatus 122 may include a cylindrical electromagnet 134, which generates the requisite static magnetic field within a bore 136 of the electromagnet 134. During medical procedures, a patient is placed inside the bore 136 on a movable support table 138. A region of interest 140 within the patient (e.g., the patient's head) may be positioned within an imaging region 142 wherein the electromagnet 134 generates a substantially homogeneous field. A set of cylindrical magnetic field gradient coils 144 may also be provided within the bore 136 and surrounding the patient. The gradient coils 144 generate magnetic field gradients of predetermined magnitudes, at predetermined times, and in three mutually orthogonal directions. With the field gradients, different spatial locations can be associated with different precession frequencies, thereby giving an MR image its spatial resolution. An RF transmitter coil 146 surrounding the imaging region 142 emits RF pulses into the imaging region 142 to cause the patient's tissues to emit magnetic-resonance (MR) response signals. Raw MR response signals are sensed by the RF coil 146 and passed to an MR controller 148 that then computes an MR image, which may be displayed to the user. Alternatively, separate MR transmitter and receiver coils may be used. Images acquired using the MRI apparatus 122 may provide radiologists and physicians with a visual contrast between different tissues and detailed internal views of a patient's anatomy that cannot be visualized with conventional x-ray technology.

The MRI controller 148 may control the pulse sequence, i.e., the relative timing and strengths of the magnetic field gradients and the RF excitation pulses and response detection periods. The MR response signals are amplified, conditioned, and digitized into raw data using a conventional image-processing system, and further transformed into arrays of image data by methods known to those of ordinary skill in the art. Based on the image data, the target region (e.g., a tumor) can be identified.

To perform targeted drug delivery, it is necessary to determine the location of the target region with high precision prior to drug administration. Accordingly, in various embodiments, the imager 122 is first activated to acquire images of the target region and/or non-target region (e.g., the healthy tissue surrounding the target region and/or the intervening tissue located between the transducer array 102 and the target region) and, based thereon, determine anatomical characteristics (e.g., a location, a size, a density, a structure and/or a shape) associated therewith. For example, a tissue volume may be represented as a 3D set of voxels (i.e., volumetric pixels) based on a 3D image or a series of 2D image slices and may include the target region and/or non-target region.

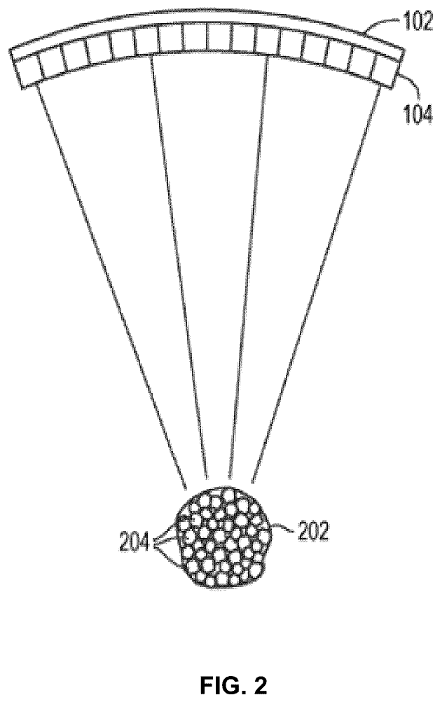

Referring to FIG. 2, in various embodiments, after the 3D voxel set corresponding to the target volume 202 is identified using the imager 122, the transducer array 102 is activated to generate a focal zone 204 in the target volume 202. Generally, the size of the target volume 202 is larger than that of the focal zone 204. Thus, the transducer array 102 may be sequentially activated to generate a plurality of focal zones 204 in the target volume 202 for disrupting the tissue therein, and thereby temporarily increasing permeability of the tissue. In addition, each focal zone 204 may be shaped (e.g., to a focal point or volume such as a sphere or a toroid) to conform to the local shape of the target region 202. Approaches to configuring the ultrasound transducer elements to generate a focal zone having a desired size and shape are provided, for example, in U.S. Pat. No. 7,611,462, the contents of which are incorporated herein by reference.

Generally, the degree of permeability and/or the size of the tissue region in which the permeability has been increased depend on the intensity and/or duration of the ultrasound application. Accordingly, by adjusting the ultrasound intensity and/or duration, the tissue permeability of the target region can be increased to a desired degree to allow the therapeutic agent to penetrate and/or diffuse therein, and monitored as described below. In some embodiments, the ultrasound procedure is adjusted based on the monitored permeability.

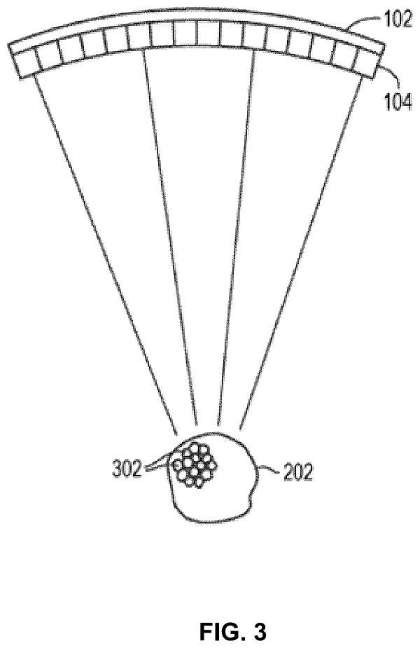

With reference to FIG. 3, in various embodiments, the ultrasonic energy emitted by the transducer elements 104 may be above a threshold and thereby cause generation of a small cloud of gas bubbles (or “microbubbles”) 302 in the liquid contained in the target region 202. The microbubbles 302 can be formed due to the negative pressure produced by the propagating ultrasonic waves or pulses, when the heated liquid ruptures and is filled with gas/vapor, or when a mild acoustic field is applied on tissue containing cavitation nuclei. At a relatively low acoustic power (e.g., 1-2 Watts above the microbubble-generation threshold), however, the generated microbubbles 302 tend to undergo oscillation with compression and rarefaction that are equal in magnitude and thus the microbubbles generally remain unruptured (i.e., a “stable cavitation”). At a higher acoustic power (e.g., more than 10 Watts above the microbubble-generation threshold), the generated microbubbles 302 undergo rarefaction that is greater than compression, which may cause inertial (or transient) cavitation of the microbubbles in which the microbubbles in the liquid rapidly collapse. The microbubble cavitation, in turn, may result in transient disruption of the tissue in the targeted region 202, and consequently increase tissue permeability in the target region. The degree of the permeability increase may depend on the microbubble concentration and/or the delivered acoustic power (or power density) and energy in the target region 202. Accordingly, a desired tissue permeability (i.e., to allow penetration/diffuse of the therapeutic agent) may be achieved by adjusting the microbubble characteristics (e.g., the concentration, administration profile, etc.) and/or ultrasound parameters (e.g., amplitude, frequency, application duration, etc.) as further described below.

In some embodiments, microbubbles are injected into the target region 202 to assist disruption of the tissue and thereby increase permeability thereof. The microbubbles may be introduced in the form of liquid droplets that subsequently vaporize, as gas-filled bubbles, or entrained with another suitable substance, such as a conventional ultrasound contrast agent. The injected microbubbles may themselves create or facilitate the creation of additional microbubbles. For example, the administration system 126 may introduce a seed microbubble into the target region 202, and the controller 108 may then cause the ultrasound waves/pulses to focus at a region proximate to the seed microbubble, thereby inducing generation of a microbubble cloud for disrupting the tissue at the target region 202. Therefore, the actual disrupting effect on the target tissue may result from a combination of the injected microbubbles and microbubbles additionally created in the tissue.

In some embodiments, the ultrasound-induced microbubble cavitation is utilized to transiently disrupt (or “open”) a targeted BBB region. Opening the BBB has been found to reduce the amyloid plaque burden, thereby providing therapeutic value for Alzheimer's disease. In addition, disrupting the BBB region may allow the therapeutic agent present in the bloodstream to penetrate the “opened” BBB region and effectively deliver therapy to the targeted brain cells. Again, the degree and size of the BBB opening may be controlled by adjusting the microbubble characteristics and/or ultrasound parameters. For example, to effectively and efficiently cause generation and/or cavitation of the microbubbles in the target region 202, it may be desirable to maximize the amount of acoustic energy transmitted to the target region 202 while minimizing the exposure of healthy non-target tissue (e.g., tissue located between the transducer and target region) to ultrasound. Typically, the degree of ultrasound absorption in tissue is a function of frequency, given by:

$$I = {I}_{0} {e}^{- 2 α fz}$$

where I₀ is the ultrasound intensity at the point of entry into the tissue (measured in W/cm²), I is the intensity after beam propagation through the tissue over a distance z (which is measured in cm), f is the frequency of the ultrasound (measured in MHz), and a is the absorption coefficient at that frequency (measured in cm⁻¹·MHz⁻¹). Higher values of the product α·f produce greater degrees of absorption in the target region but also larger fractions of ultrasound that are absorbed before reaching the target region. Therefore, at a certain depth, z, in tissue, the ultrasound frequency of the applied waves may reflect a trade-off between the absorption of the acoustic power in the path zone and the peak intensity at the focal zone. In some embodiments, an optimal ultrasound transmission frequency is determined based on the anatomical characteristics (e.g., type, size, location, property, structure, thickness, density, etc.) of the target and/or intervening tissue so as to achieve a peak intensity at the target region 202. Based thereon, the transducer elements can then be activated to cause generation and/or cavitation of the microbubbles. Approaches to determining an optimal frequency for the ultrasound application are provided, for example, in U.S. Patent Publication No. 2016/0008633, the contents of which are incorporated herein by reference.

In addition, if the microbubbles are pre-formed and introduced to the target volume 202 via the administration system 106, it may be desirable to select a size distribution thereof such that the microbubble resonance frequency differs from the ultrasound transmission frequency for avoiding damage of the non-target region. Generally, the smaller the radius of the microbubbles, the larger will be their resonance frequency. Accordingly, once the optimal ultrasound frequency is determined, the mean radius of microbubbles having a resonance frequency substantially equal to the ultrasound frequency may be determined. In one implementation, the size distribution of the pre-formed microbubbles is selected such that a significant fraction (e.g., more than 50%, 90%, 95%, or 99% or more) of the microbubbles have a radius below that corresponding to a resonance frequency equal to the applied ultrasound frequency. Preferably, the microbubble resonance frequency is substantially larger than the ultrasound frequency (e.g., by a factor of ten), but it can be substantially smaller than the ultrasound frequency, if desired. As a result, when the transducer elements 104 are activated with a low acoustic power, microbubbles at the non-target region are unresponsive to the relatively low acoustic field, whereas microbubbles at the target region (where the acoustic field is relatively high due to the focused beam) 202 may oscillate and/or collapse. Accordingly, this approach may disrupt tissue at the target volume 202 with high spatial accuracy and avoid undesired collateral damage to the healthy tissue surrounding the target.

The ultrasound controller 108 and/or MR controller 148 may include one or more modules implemented in hardware, software, or a combination of both. For embodiments in which the functions are provided as one or more software programs, the programs may be written in any of a number of high level languages such as PYTHON, FORTRAN, PASCAL, JAVA, C, C++, C#, BASIC, various scripting languages, and/or HTML. Additionally, the software can be implemented in an assembly language directed to the microprocessor resident on a target computer; for example, the software may be implemented in Intel 80×86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

The therapeutic agent may include any drug that is suitable for treating a tumor. For example, for treating glioblastoma (GBM), the drug may include or consist of, e.g., one or more of Busulfan, Thiotepa, CCNU (lomustine), BCNU (carmustine), ACNU (nimustine), Temozolomide, Methotrexate, Topotecan, Cisplatin, Etoposide, Irinotecan/SN-38, Carboplatin, Doxorubicin, Vinblastine, Vincristine, Procarbazine, Paclitaxel, Fotemustine, Ifosfamide/4-Hydroxyifosfamide/aldoifosfamide, Bevacizumab, Nimotuzumab, 5-Fluorouracil, Bleomycin, Hydroxyurea, Docetaxel, Cytarabine (cytosine arabinoside, ara-C)/ara-U, etc.

In addition, for treating GBM, those skilled in the art can select a drug and a BBB opening regime optimized to enhance drug absorption across the BBB within patient safety constraints. In this regard, it is known that the BBB is actually already disrupted in the core of many tumors, allowing partial penetration of antitumor drugs; but the BBB is widely intact around the “brain adjacent to tumor” (BAT) region where invasive/escaping GBM cells can be found, and which cause tumor recurrence. Overcoming the BBB for better drug delivery within the tumor core and the BAT can be accomplished using ultrasound as described herein. The drugs employed have various degrees of toxicity and various penetration percentages through the BBB. An ideal drug has high cytotoxicity to the tumor and no BBB penetration (so that its absorption and cytotoxic effects can be confined to regions where the BBB is disrupted), low neurotoxicity (to avoid damage to the nervous system), and tolerable systemic toxicity (e.g., below a threshold) at the prescribed doses. The drug may be administered intravenously or, in some cases, by injection proximate to the tumor region. In addition, configurations of the administration system 106 for introducing microbubbles and/or therapeutic agent into the target region 202 may be found in U.S. Patent Application No. 62/597,076, the contents of which are incorporated herein by reference.

### 2. Monitoring Tissue Permeability

As explained above, the observed relationship between the Rz parameter and the amount of injected drug that reaches the target area following ultrasound-induced BBB disruption can be used to track the progress of treatment. An indicator parameter based on, for example, Rz can be measured during the ultrasound treatment and the measurement used to estimate drug penetration to the target area without the need for separate MR acquisition.

In particular, the ΔR₂* parameter is typically used as a safety indicator during ultrasound treatments. The ΔR₂* parameter can also serve as an index indicating the degree of BBB disruption, enabling estimation of its permeability to drugs having a given size range. Changes in R₂* values are directly related to the total amount of deoxyhemoglobin in the tissue. Since hemoglobin is a large protein (64 kDa tetramers) that can be extravasated due to hemolysis without causing damage to blood vessels during the BBB treatments, changes in R₂* values can be correlated to the concentration of large molecules.

Liposomes are herein used here as a “model molecule” for ease of explanation, but it should be understood that other molecules or molecular complexes, such as quantum dots or dextran, may also or alternatively be used. Liposomes may incorporate Gd ions to be traceable and measurable by MRI and may be produced in different clinically relevant sizes. Polyethylene glycol (PEG)-based liposome nanoparticles may be used because of their clinical relevance as nano-carriers and due to their slow metabolic decay, which can stabilize their parenchymal concentration following BBB traversal. Indeed, during experiments, the parenchymal presence of Gd-liposomes were detected in rats several hours after injection and remained stable for at least one month following closure of the BBB. They can potentially linger for even longer periods of time, providing a useful platform for sustained drug delivery in the brain.

R₂* and R₁ maps generated from pre- and post-treatment MR data may serve as the basis for ΔR₂* and Gd-liposomal concentration maps useful in treatment. This enables establishment of an empirical relationship between ΔR₂* values and Gd-liposomal concentrations, considering the diffusion process of liposomes following their brain parenchyma entrance. This relationship, in turn, may serve as an effective proxy for the penetration of a similarly sized therapeutic of interest under the same sonication conditions. Different statistical BBB transfer dynamics may occur for different sized liposomes as a function of ΔR₂*. It is found that the smaller the nanoparticle, the higher its parenchymal concentration will be for the same ΔR₂* values. Treatment regions may include size-dependent concentration plateau regions, where the parenchymal liposome concentrations are independent of ΔR₂*. The larger the nanoparticle, the higher its ΔR₂* threshold dependence will be.

ΔR₂* can be measured during or immediately after treatment using a conventional 1.5T MRI device, without requiring radioactive tracers, long measurement time or strong magnetic fields, while preserving an adequate signal-to-noise ratio. ΔR₂* signals can predict BBB traversal by differently sized molecules, enabling effective trans-BBB therapies.

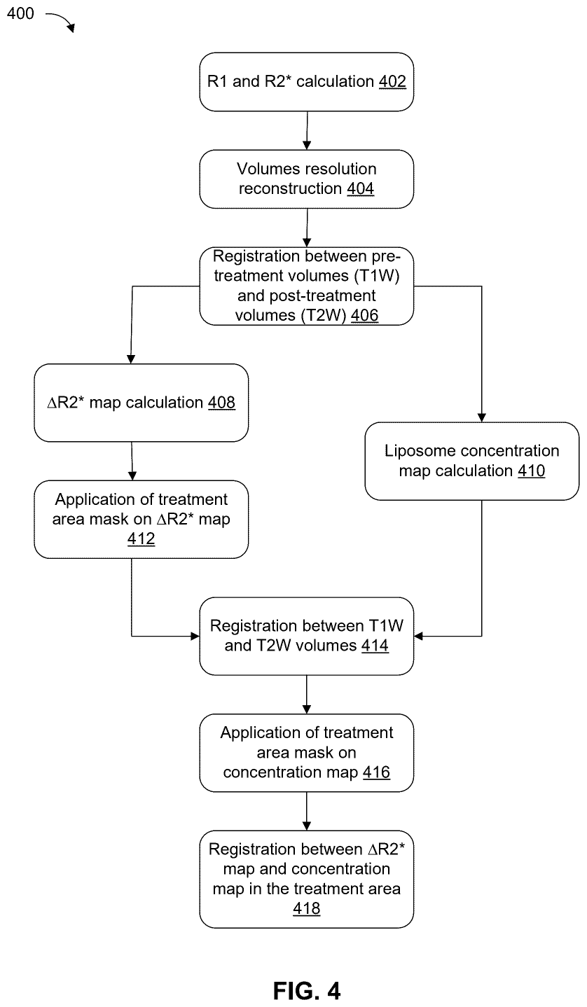

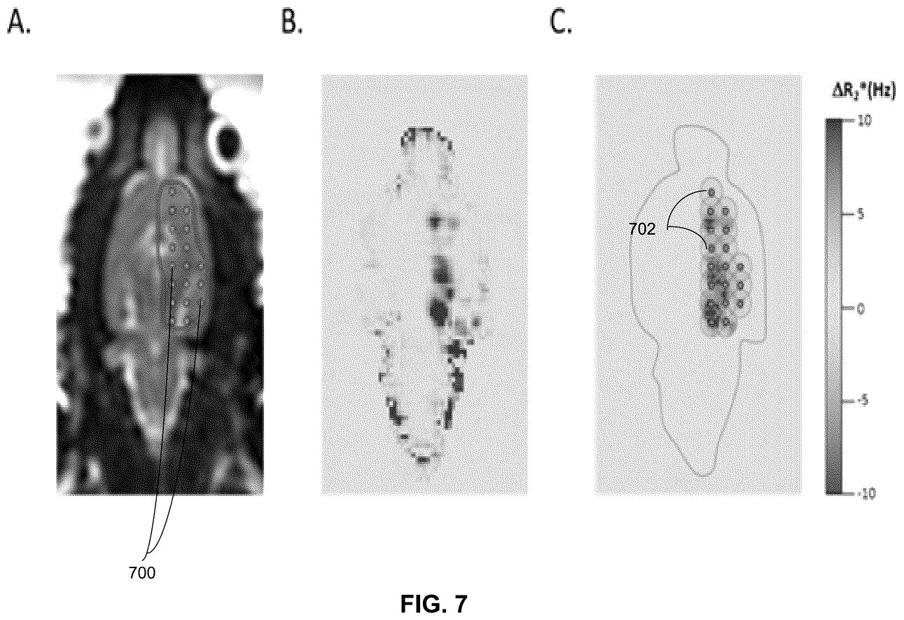

FIG. 4 illustrates a representative method 400 for relating an MR signal to penetration of liposomes across the BBB. The analysis starts with per-voxel computation of ΔR₂* and R₁ in pre- and post-treatment volumes (step 402), followed by volumetric reconstruction of the ΔR₂* and R₁ voxels (of size 0.5 mm3) using linear interpolation (step 404). This is followed by a registration step (406), which computes a registration transformation (e.g., rigid then affine) between the pre- and post-treatment T1 weighted volume (T1W) and the pre- and post-treatment T₂ weighted volume (T2W). ΔR₁ and ΔR₂* values may be obtained from, respectively, T1W and T2W volumes (step 408) by voxelwise extraction of T1 and T2* relaxation times from pre- and post-treatment fast spin echo volumes with different repetition times (TRs), and pre- and post-treatment gradient echo volumes with different time to echo (TE) values, respectively. The ΔR₁ values may be translated into Gd-liposome concentration (step 410) using, for example, a calibration curve as described below. The liposome distribution tracks the ΔR₂* signal, so a treatment mask indicative of the liposome distribution may be extracted from the ΔR₂* map (step 412) using subspot locations (an example of which is shown in FIG. 7 described below). Maps of the T₁ and T₂ weighted volumes T1W and T2W are then registered (step 414) and used as a mask, which is applied to the Gd-liposome map created in step 412 to isolate concentration changes related to the treated area (step 416). To model liposome diffusion, the ΔR₂* signal map and the Gd-liposomal concentration map are compared to find the geometric relationship between the two parameters (step 418).

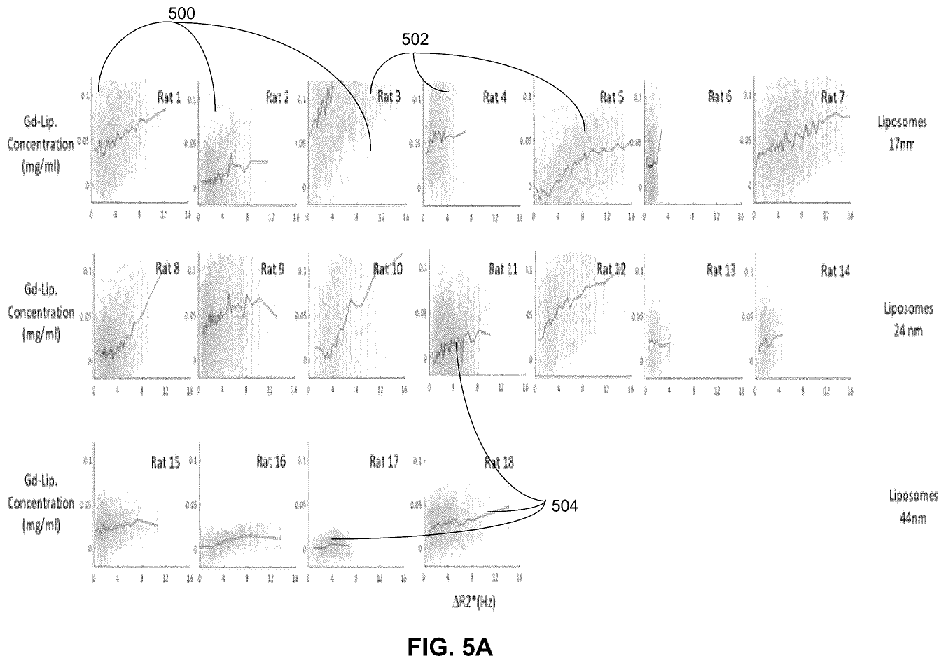

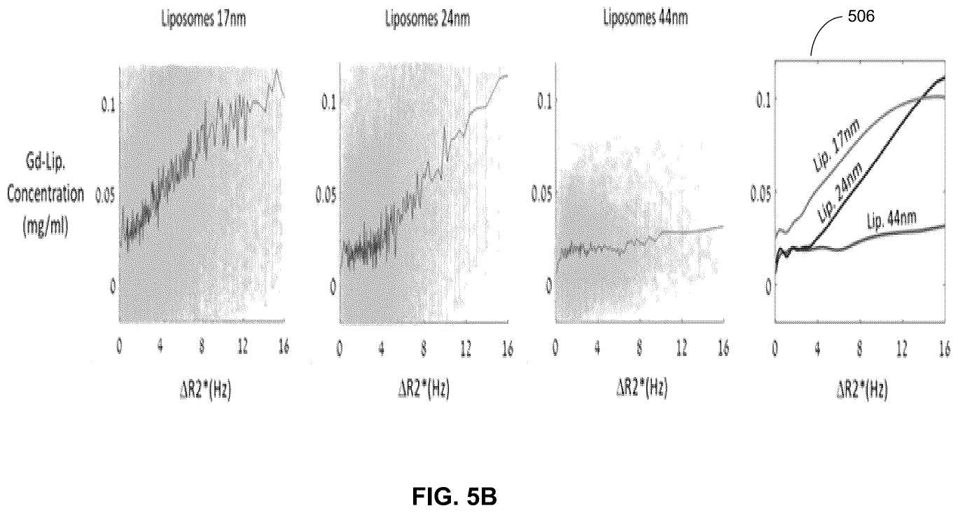

FIGS. 5A and 5B representatively illustrate the relationship between ΔR₂* and Gd-liposomal concentration in accordance with various embodiments. FIG. 5A shows voxelwise representations of different Gd-liposome populations in a rat model. The circles 500 are the voxelwise data points, the curves 504 are the averages of every 100 adjacent points, and the bars 504 are standard deviations. The differences observed among rats is noticeable. FIG. 5B shows same analysis as in FIG. 5A, but with pooled data from all rats in the population. The graph plot 506 contains interpolation curves based on the three plots to the left. These plots indicate that smaller liposomes have higher parenchymal concentrations for the same ΔR₂* values, and larger liposomes exhibit higher ΔR₂* threshold dependence.

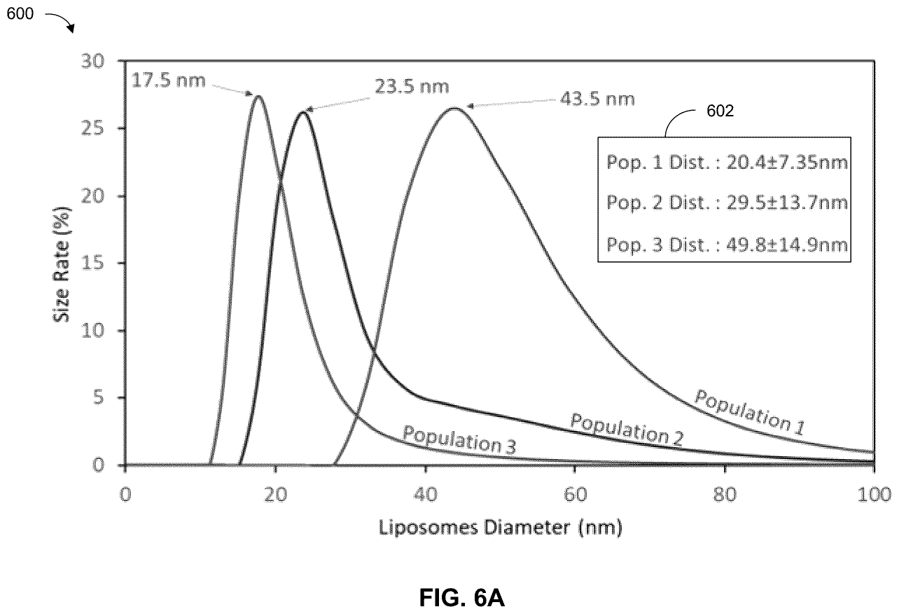

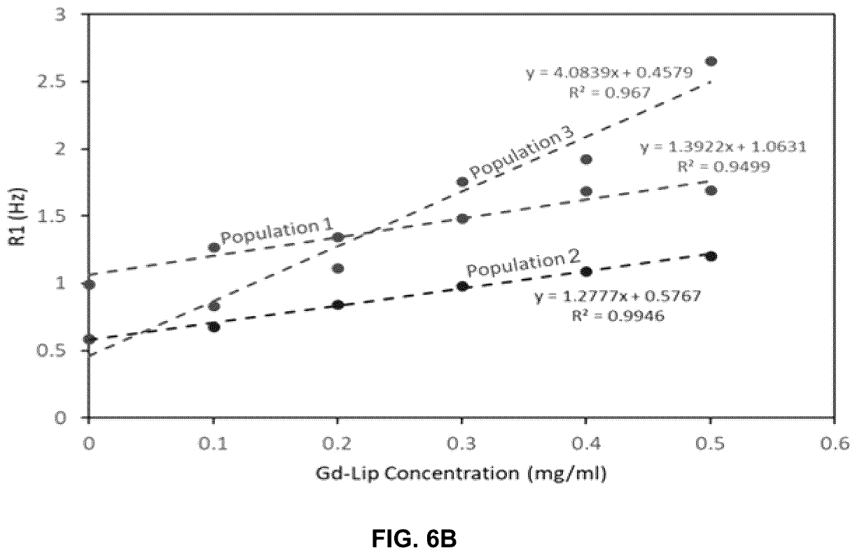

FIG. 6A shows a plot 600 of particle size distribution expressed as a frequency, calculated from DLS data, with the average and standard deviation of each population indicated at 602. The numbers above the curves (17.5 nm, 23.5 nm, and 43.5 nm) indicate the distribution peaks.

FIG. 6B shows a graph plot 604 of R₁ against the concentration of Gd-tagged liposomes. The calibration was performed using an array of polycarbonate wells, each containing 0.5 ml of rat blood with different Gd-liposome concentrations. To maintain a consistent R₁ baseline, the rat blood content in each liposome population measurement remained the same. Each calibration was performed on a single rat's blood. Population 1 corresponds to 17 nm liposomes, Population 2 corresponds to 24 nm liposomes, and Population 3 corresponds to 44 nm liposomes.

As noted above, a treatment mask indicative of the liposome distribution may be extracted from the ΔR₂* map using subspot locations. These are visible manifestations of the treatment-related ΔR₂* signals. The subspots may be used as a mask to isolate liposome regions, the sizes of which indicate numbers of liposomes and permit computation of the liposome concentration given the remaining volume. This is illustrated in FIGS. 7A-7C. FIG. 7A indicates the locations of subspots 700 in the T2W maps. FIG. 7B shows the relationship between ΔR₂* values in the treatment area and the sonication targets; in particular, the circles shown in FIG. 7C have diameters corresponding to the lateral spot width (e.g., 3 mm) of the ultrasound beam. Assuming that liposomes undergo diffusion once they enter the brain parenchyma, another affine registration may be performed between ΔR₂* and the subspot map to track the diffusion. Finally, the liposomal concentration may be plotted as function of the ΔR₂* signal on voxelwise basis.

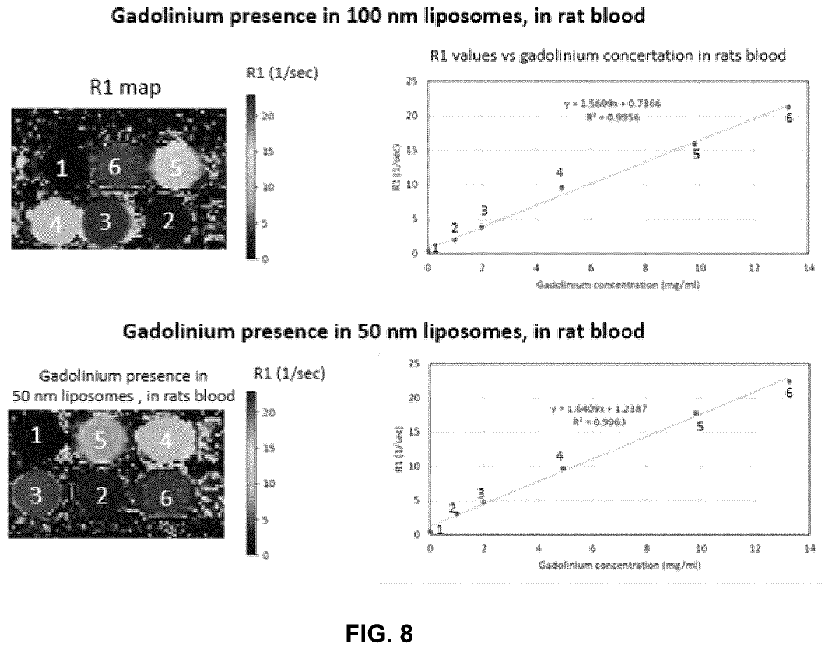

As indicated in FIGS. 8A and 8B, R₁ signals may be similarly be employed. FIG. 8A depicts an R₁ map indicating the presence of Gd in 100 nm liposomes and a graphical plot of R₁ against Gd concentration in rat blood; and FIG. 8B shows similar results for 50 nm liposomes. For both maps and graphs, measurements of ΔR₁ were obtained using tubes filled with Gd liposomes in known concentration (the tubes covered range of concentrations) to correlate ΔR₁ to the liposome concentration. After registration as described above, the ΔR₂* value was derived for each pixel from intraoperative T₂* images, and ΔR₁ values were derived from post-treatment images. The ΔR₁ values were converted to concentration values based on actual measurements, thereby correlating pixel-level ΔT₂* values with the associated liposome concentration. This produced the calibration curves 804 and 806, which relate ΔR₁ values to liposome concentration.

### 3. Methods of Disrupting Target Tissue for Treatment

Provided herein in various aspects is a method for disrupting target tissue for treatment. In various embodiments, the method includes sonicating the target volume to cause disruption of the target tissue therein and thereby increase a permeability thereof, monitoring a degree of permeability of the target tissue caused by the disruption; and ceasing sonication of the target volume when the determined permeability reaches a threshold. In some embodiments, the ultrasound system 100 of any of the embodiments disclosed herein may be used for generating and delivering a focused acoustic energy beam to the target region for disrupting the tissue and thereby causing the tissue permeability to increase therein.

In some embodiments, the degree of permeability is calculated based on a measurement of a detection marker inside the target tissue, wherein the detection marker has been administered to a subject in which the degree of permeability is being monitored.

In some embodiments, the molecules that are used to measure the degree of permeability are detection markers that are administered to a subject. In these embodiments, the measurement of the detection markers in the target tissue is indicative of transit of the detection markers from the blood to the target tissue upon application of ultrasound. In these embodiments, detection and measurement of these detection markers inside the target tissue after the induction tissue permeability is used for calculation of transit of therapeutic agents having molecular weights similar to that of the detection markers. In these embodiments, detection and measurement of these detection markers inside the target tissue after the induction tissue permeability is used for normalization for comparison of the transit of therapeutic agents having molecular weights similar to that of the biomarker between different applications of induction of tissue permeability. In some embodiments, the detection marker is a MRI-visible contrast agent. In some embodiments, the MRI-visible contrast agent comprises ionic Gd). In some embodiments, the detection marker is a positron emission tomography (PET) reporter. In some embodiments, the PET reporter is selected from 18F-florbetapir, 18F-Florbetaben, 18F-Flortaucipir, 18F-Flutemetamol and 18F-Flourodopa (F-Dopa). In some embodiments, the detection marker is a single photon emission computed tomography (SPECT) reporter. In some embodiments, the SPECT reporter is selected from 99mTc-ECD (ethylcysteinate-dimer), 123I-Ioflupane, and 99mTc-TRODAT-1). The size of these agents may be adjusted by chemically conjugating with an entity such as a polymer (without limitation, e.g., dextran and polyethylene glycol), or by complexing with a large complex such as a liposome or a quantum dot.

In some embodiments, the detection marker is conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is a MRI-visible contrast agent, which optionally comprises ionic Gd. In some embodiments, sonication comprises at least two applications on different days in the same patient. In some embodiments, sonication comprises different applications in different patients.

In some embodiments, the degree of permeability is calculated based on the measurement in a biological sample from the subject in which the degree of permeability is being monitored of a level of a molecule that is predominantly present in the target tissue but substantially not present in tissues outside the target tissue under normal conditions. In some embodiments, the target tissue is brain. In some embodiments, the biological sample is blood. In some embodiments, the molecule that is predominantly present in the target tissue but substantially not present in tissues outside the target tissue under normal conditions is a brain protein. In some embodiments, the brain protein is not usually found outside a cranial tissue (e.g., in the bloodstream). In some embodiments, the brain protein leaks outside a cranial tissue (e.g., in the bloodstream) when the blood-brain barrier is opened sufficiently to allow the leakage of molecules having size or molecular weight of the brain protein. In some embodiments, the brain protein becomes detectable in a biological sample (e.g. a blood sample) when the blood-brain barrier is opened sufficiently to allow the leakage of molecules having size or molecular weight of the brain protein. Illustrative, non-limiting examples of molecules that are predominantly present in the target tissue but substantially not present in tissues outside the target tissue include the brain proteins including, but not limited to, NSE, S100B, GFAP and UCH-L1, MBP, tau, and NfL. Murcko et al., Diagnostic biomarker kinetics: how brain-derived biomarkers distribute through the human body, and how this affects their diagnostic significance: the case of S100B. Fluids Barriers CNS 2022; 19: 32; Janigro et al., Peripheral Blood and Salivary Biomarkers of Blood-Brain Barrier Permeability and Neuronal Damage: Clinical and Applied Concepts, Front Neurol. 2021; 11:577312. Native form of NSE is homodimeric with a molecular weight of about 78 kDa. See Pihlman et al., Purification and characterization of human neuron-specific enolase: radioimmunoassay development, Tumour Biol 1984; 5(2): 127-39. S100B is also a homodimeric protein of 21.5 kDa, although homotetrameric and homohexameric forms are known. See Thulin et al., Molecular determinants of S100B oligomer formation, PLoS One 2011; 6(3):e14768. Accordingly, In some embodiments, the species of NSE that is detected has a molecular weight of about 78 kDa. In some embodiments, the species of S100B that is detected has a molecular weight of about 22 kDa. In some embodiments, the species of GFAP that is detected is a monomer (50 kDa), a dimer (100 kDa) and/or a tetramer (200 kDa). Tang et al., J Biol Chem. Oligomers of Mutant Glial Fibrillary Acidic Protein (GFAP) Inhibit the Proteasome System in Alexander Disease Astrocytes, and the Small Heat Shock Protein αB-Crystallin Reverses the Inhibition, 2010; 285(14): 10527-10537. Likewise, in some embodiments, the species of UCH-L1, MBP, tau, and NfL have molecular weights of about 25 kDa, about 18,500 Daltons, about 62 kDa, and about 68 kDa, respectively.

In some embodiments, sonication comprises at least two applications on different days in the same patient. In some embodiments, sonication comprises different applications in different patients.

In some embodiments, the molecules that are used to measure the degree of permeability are biomarkers specific to target tissue. In these embodiments, the measurement of the detection markers outside the target tissue is indicative of transit of the biomarkers from the target tissue to outside the target tissue (without limitation, e.g., in blood) upon application of ultrasound. In these embodiments, detection and measurement of these molecules outside the target tissue after the induction tissue permeability is used for calculation of transit of therapeutic agents having molecular weights similar to that of the molecules. In these embodiments, detection and measurement of these molecules outside the target tissue after the induction tissue permeability is used for normalization for comparison of the transit of therapeutic agents having molecular weights similar to that of the biomarker between different applications of induction of tissue permeability. In some embodiments, the target tissue-specific biomarkers include the central nervous tissue-specific markers such as the NSE and S100B, which are not usually found in blood. In some embodiments, the species of NSE that is detected has a molecular weight of about 78 kDa. In some embodiments, the species of S100B that is detected has a molecular weight of about 22 kDa. In some embodiments, the species of NSE that is detected has a molecular weight of about 78 kDa. In some embodiments, the species of S100B that is detected has a molecular weight of about 22 kDa. In some embodiments, the species of GFAP that is detected is a monomer (50 kDa), a dimer (100 kDa) and/or a tetramer (200 kDa). Tang et al., J Biol Chem. Oligomers of Mutant Glial Fibrillary Acidic Protein (GFAP) Inhibit the Proteasome System in Alexander Disease Astrocytes, and the Small Heat Shock Protein αB-Crystallin Reverses the Inhibition, 2010; 285(14): 10527-10537. Likewise, in some embodiments, the species of UCH-L1, MBP, tau, and NfL have molecular weights of about 25 kDa, about 18,500 Daltons, about 62 kDa, and about 68 kDa, respectively. Accordingly, in some embodiments, the measurement of the species of NSE is used for normalization of transit of a therapeutic agent in the molecular weight range of about 60 to about 100 kDa. In some embodiments, the measurement of the species of S100B is used for normalization of transit of a therapeutic agent in the molecular weight range of about 15 to about 30 kDa. In some embodiments, the measurement of a species of GFAP is used for normalization of transit of a therapeutic agent in the molecular weight range of about 40 to about 250 kDa, depending on the species detected. In some embodiments, the measurement of a species of UCH-L1 is used for normalization of transit of a therapeutic agent in the molecular weight range of about 20 to about 30 kDa. In some embodiments, the measurement of a species of the MBP is used for normalization of transit of a therapeutic agent in the molecular weight range of about 15 to about 20 kDa. In some embodiments, the measurement of a species of tau is used for normalization of transit of a therapeutic agent in the molecular weight range of about 50 to about 70 kDa. In some embodiments, the measurement of a species of NfL is used for normalization of transit of a therapeutic agent in the molecular weight range of about 60 to about 80 kDa. Accordingly, in various embodiments, the measurement of the species of one or more of the molecules that are predominantly present in the target tissue but substantially not present in tissues outside the target tissue is used for normalization of transit of a therapeutic agent in the molecular weight range of about 15 to about 250 kDa.

### 4. Methods of Comparing the Degree of Permeability of a Target Tissue Among at Least Two Applications

Provided herein in various aspects is a method for comparing a degree of permeability of a target tissue among at least two applications of sonication. In various embodiments, the method comprises providing a biological sample from the subject that received a first application of sonication to a target volume to cause disruption of the target tissue therein and thereby increase a permeability thereof. In some embodiments, the target tissue is brain and/or the biological sample is blood. In some embodiments, the method further comprises measuring the amount in the biological sample of a molecule that is predominantly present in the target tissue but substantially not present in tissues outside the target tissue under normal conditions. In some embodiments, the measurement of the molecule may be performed using any technique known to skilled persons. In some embodiments, the method further comprises monitoring a degree of permeability of the target tissue caused by the disruption. In some embodiments, the method further comprises providing a second biological sample from the subject that received a second application of sonication on a different day to a target volume to cause a second disruption of the target tissue therein and thereby increase a second permeability thereof. In some embodiments, the method further comprises measuring in the second biological sample the amount of the molecule that is predominantly present in the target tissue but substantially not present in tissues outside the target tissue under normal conditions. In some embodiments, the method further comprises monitoring a degree of permeability of the target tissue caused by the second disruption. In some embodiments, the method further comprises comparing a degree of permeability of the target tissue in a subject among at least two applications of sonication on different days.

In some embodiments, the target tissue is brain and/or the biological sample is blood. In some embodiments, the molecule that is predominantly present in the target tissue but substantially not present in tissues outside the target tissue under normal conditions. Illustrative, non-limiting examples of molecules that are predominantly present in the target tissue but substantially not present in tissues outside the target tissue include, but are not limited to, the brain proteins NSE, S100B, GFAP and UCH-L1, MBP, tau, and NfL. In some embodiments, the species of NSE that is detected has a molecular weight of about 78 kDa. In some embodiments, the species of S100B that is detected has a molecular weight of about 22 kDa. In some embodiments, the species of NSE that is detected has a molecular weight of about 78 kDa. In some embodiments, the species of S100B that is detected has a molecular weight of about 22 kDa. In some embodiments, the species of GFAP that is detected is a monomer (50 kDa), a dimer (100 kDa) and/or a tetramer (200 kDa). Tang et al., J Biol Chem. Oligomers of Mutant Glial Fibrillary Acidic Protein (GFAP) Inhibit the Proteasome System in Alexander Disease Astrocytes, and the Small Heat Shock Protein αB-Crystallin Reverses the Inhibition, 2010; 285(14): 10527-10537. Likewise, in some embodiments, the species of UCH-L1, MBP, tau, and NfL have molecular weights of about 25 kDa, about 18,500 Daltons, about 62 kDa, and about 68 kDa, respectively. Accordingly, in some embodiments, the measurement of the species of NSE is used for normalization of transit of a therapeutic agent in the molecular weight range of about 60 to about 100 kDa. In some embodiments, the measurement of the species of S100B is used for normalization of transit of a therapeutic agent in the molecular weight range of about 15 to about 30 kDa. In some embodiments, the measurement of a species of GFAP is used for normalization of transit of a therapeutic agent in the molecular weight range of about 40 to about 250 kDa, depending on the species detected. In some embodiments, the measurement of a species of UCH-L1 is used for normalization of transit of a therapeutic agent in the molecular weight range of about 20 to about 30 kDa. In some embodiments, the measurement of a species of the MBP is used for normalization of transit of a therapeutic agent in the molecular weight range of about 15 to about 20 kDa. In some embodiments, the measurement of a species of tau is used for normalization of transit of a therapeutic agent in the molecular weight range of about 50 to about 70 kDa. In some embodiments, the measurement of a species of NfL is used for normalization of transit of a therapeutic agent in the molecular weight range of about 60 to about 80 kDa. Accordingly, in various embodiments, the measurement of the species of one or more of the molecules that are predominantly present in the target tissue but substantially not present in tissues outside the target tissue is used for normalization of transit of a therapeutic agent in the molecular weight range of about 15 to about 250 kDa.

In some embodiments, the molecule that is predominantly present in the target tissue but substantially not present in tissues outside the target tissue under normal conditions is a tumor-derived marker such as circulating tumor cells, circulating tumor DNA, tumor extracellular vesicles, etc. In some embodiments, the molecule that is predominantly present in the target tissue but substantially not present in tissues outside the target tissue under normal conditions is a tumor-derived marker such as circulating tumor cells, circulating tumor DNA, tumor extracellular vesicles, etc. In some embodiments, the molecule that is predominantly present in the target tissue but substantially not present in tissues outside the target tissue under normal conditions is a biomarker for stroke, including, but not limited to, S100B, NSE, MBP, and GFAP. In some embodiments, the molecule that is predominantly present in the target tissue but substantially not present in tissues outside the target tissue under normal conditions is a biomarker for neurodegenerative disorders, including, but not limited to, axonal protein and NfL. In some embodiments, the molecule that is predominantly present in the target tissue but substantially not present in tissues outside the target tissue under normal conditions is a biomarker for Alzheimer's disease (AD), including, but not limited to, phosphorylated tau proteins P-tau181 and P-tau217. In some embodiments, the molecule that is predominantly present in the target tissue but substantially not present in tissues outside the target tissue under normal conditions is a biomarker for traumatic brain injuries, including, but not limited to, NfL, UCH-L1, tau, S100B, and GFAP.

In some embodiments, the measurement of the molecule that is predominantly present in the target tissue but substantially not present in tissues outside the target tissue under normal conditions is performed using any technique known to skilled persons. In some embodiments, the measurement of the molecule that is predominantly present in the target tissue but substantially not present in tissues outside the target tissue under normal conditions is performed using mass spectrometry (without limitation, e.g., surface-enhanced Raman spectroscopy (SERS), and UltraSEEK®). In some embodiments, the measurement of the molecule that is predominantly present in the target tissue but substantially not present in tissues outside the target tissue under normal conditions is performed using an immunological technique such as enzyme-linked immunosorbent assay (ELISA), radioimmune assay (RIA), fluorescence immunoassays, and chemoluminescence immunoassays. In some embodiments, the measurement of the molecule that is predominantly present in the target tissue but substantially not present in tissues outside the target tissue under normal conditions is performed using a technique that detects a nucleic acid, including but not limited to quantitative PCR (Q-PCR), Droplet Digital polymerase chain reaction (ddPCR), methylation-specific PCR (MS-PCR), Beads, Emulsion, Amplification and Magnetics (BEAMing), Safe-Sequencing System (Safe-SeqS), Cancer Personalized Profiling by deep sequencing (CAPP-Seq), Tagged-amplicon deep sequencing (TAm-Seq), whole-genome sequencing (WGS), or whole exome sequencing (WES)

In another aspect, a method is provided for comparing a degree of permeability of a target tissue in a subject among at least two applications of focused ultrasound treatment on different days. In some embodiments, the method comprises providing a subject that received a first application of sonication to a target volume to cause disruption of the target tissue therein and thereby increase a permeability thereof. In some embodiments, the subject has received a detection marker. In some embodiments, the subject has received the detection marker on the same day. In some embodiments, the method further comprises measuring the level of the detection marker in the target tissue, and thereby monitoring a degree of permeability of the target tissue caused by the disruption. In some embodiments, the method further comprises providing a second biological sample from the subject that received a second application of sonication on a different day to a target volume to cause a second disruption of the target tissue therein and thereby increase a second permeability thereof, optionally wherein the subject has received a detection marker on the same day. In some embodiments, the method further comprises measuring the level of the detection marker in the target tissue, and thereby monitoring a degree of permeability of the target tissue caused by the second disruption, and thereby monitoring a degree of permeability of the target tissue caused by the second disruption. In some embodiments, the method further comprises comparing a degree of permeability of the target tissue in a subject among at least two applications of sonication on different days.

In some embodiments, the target tissue is brain. In some embodiments, the target tissue is brain and the detection marker is conjugated with a polymer (e.g., a dextran or a polyethylene glycol) that is in a defined molecular weight range corresponding to the molecular weight range of a therapeutic agent. In some embodiments, the target tissue is brain and the detection marker is complexed a liposome having a defined size range corresponding to the size range of a therapeutic agent. In some embodiments, the detection marker is a MRI-visible contrast agent. In some embodiments, the MRI-visible contrast agent comprises ionic Gd. In some embodiments, the detection marker is a positron emission tomography (PET) reporter. In some embodiments, the PET reporter is selected from 18F-florbetapir, 18F-Florbetaben, 18F-Flortaucipir, 18F-Flutemetamol and 18F-Flourodopa (F-Dopa). In some embodiments, the detection marker is a single photon emission computed tomography (SPECT) reporter. In some embodiments, the SPECT reporter is selected from 99mTc-ECD (ethylcysteinate-dimer), 123I-Ioflupane, and 99mTc-TRODAT-1).

It should be understood that calibration may be achieved in any conventional manner, and may involve machine-learning techniques, linear or nonlinear regression, or other suitable technique. Furthermore, following calibration, the controller 108 may be configured or programmed to control the ultrasound treatment dynamically, in response to ongoing estimates of permeability.

It is found that, unlike low-weight Gd-based contrast agents that are totally washed out from the parenchyma several hours after administration, liposomes can be detected in the brain several hours post-injection and stay in the tissue for at least for one month following administration. This phenomenon can be explained by the size of the liposomes, which are an order of magnitude larger than Gd salts, thus complicating their transfer through the BBB; and by the long (tens of hours) half-life of PEG-based liposomes in the blood, which allows the liposomes to accumulate in the parenchyma and slow down the washout process to final stabilization of its parenchymal concentration with closure of the BBB. As a result, the liposome concentration may be determined at least in part by either a wash-in or a wash-out time. In particular, high wash-in—wash-out times correspond to high permeabilities, as molecules traverse the BBB quickly.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

5. The Neurological Disease or Disorders that May be Treated Using the Method and System Disclosed Herein

In some aspects, the present disclosure provides a method of treating a neurological disease or disorder in a subject in need thereof, wherein the neurological disease or disorder is characterized by abnormal production, aggregation, and/or deposition of a protein or another biomolecule in the brain. In some embodiments, neurological disease or disorder is selected from the Alzheimer's Disease (AD), Parkinson's Disease (PD), Huntington's Disease (HD), amyotrophic lateral sclerosis (ALS), dementia with Lewy bodies, spinocerebellar ataxia, and amyotrophic lateral sclerosis, frontotemporal diseases, multiple system atrophy, four-repeat tauopathy and prion diseases.

Alzheimer's Disease patients exhibit senile plaques that are mainly composed of amyloid-β (Aβ), neurofibrillary tangles, which include Tau protein, neuronal inclusions of TDP-43 as well as Lewy bodies, which include α-Synuclein. Parkinson's Disease patients exhibit Lewy bodies, which include α-Synuclein. Patients suffering from amyotrophic lateral sclerosis have neuronal inclusions that include TAR DNA-binding protein 43 (TDP-43), fused in sarcoma/translocated in liposarcoma (FUS/TLS), and superoxide dismutase-1 (SOD1). Huntington's Disease is a progressive brain disorder caused by a mutation in the gene coding for the huntingtin protein, resulting in an abnormal mutant protein that gradually damages brain cells. Dementia with Lewy bodies features Lewy bodies, which include α-Synuclein, senile plaques that are mainly composed of amyloid-β (Aβ) and neurofibrillary tangles of Tau protein. The patients having frontotemporal diseases show neuronal and glial inclusions composed of Tau, TDP-43, and FUS/TLS. Multiple system atrophy features glial cytoplasmic inclusions of α-synuclein. Thus, there appears to be an overlap between the proteins that exhibits abnormal production, aggregation, and/or deposition associated with these diseases. A single neurodegenerative disease can be associated with multiple proteins (or another biomolecules) that exhibits abnormal production, aggregation, and/or deposition. On the other hand, a single the protein that exhibits abnormal production, aggregation, and/or deposition can also be associated with multiple diseases. For example, although Aβ plaques and tau tangles are paradigmatic of Alzheimer's Disease, Lewy bodies typical of Parkinson's Disease are found in more than 50 percent of Alzheimer's cases, and neuronal inclusions consisting of the protein TDP-43 are found in more than 40 percent. Similarly in dementia with Lewy bodies, a dementing disorder closely allied to Parkinson's disease having some features of Alzheimer's, the paradigmatic α-Synuclein-rich Lewy bodies are accompanied by Aβ plaques in 60 percent of cases and tau tangles in 50 percent. Likewise, four-repeat tauopathies, a group of neurodegenerative diseases defined by cytoplasmic inclusions predominantly composed of tau protein isoforms with four microtubule-binding domains, is associated with at least three clinical presentations: (1) progressive supranuclear palsy presents with an axial rigidity and eye movement problems, in addition to atypical Parkinsonism; (2) corticobasal degeneration presents like a frontal lobe dementia, with focal cortical syndromes, including progressive apraxia or progressive aphasia; and (3) argyrophilic grain disease is an increasingly recognized disorder of the elderly that affects the medial temporal lobe and is associated with an amnesic cognitive impairment.

Accordingly, in some aspects, the present disclosure provides a method of treating a neurological disease or disorder in a subject in need thereof, wherein the neurological disease or disorder is characterized by abnormal production, aggregation, and/or deposition of a protein or another biomolecule in the brain, wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control.

In another aspect, the invention relates to a method of treating a neurological disease or disorder in a subject in need thereof, wherein the neurological disease or disorder is characterized by having a locus of abnormal production, aggregation, and/or deposition of a protein or another biomolecule in the brain, the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition. In some embodiments, the neurological disease or disorder is Alzheimer's Disease and the locus is selected from a senile plaque comprising amyloid-β (Aβ), neurofibrillary tangles comprising Tau protein, neuronal inclusions comprising TDP-43, and Lewy bodies comprising α-Synuclein. In some embodiments, the neurological disease or disorder is Parkinson's Disease and the locus is Lewy bodies comprising α-Synuclein. In some embodiments, the neurological disease or disorder is amyotrophic lateral sclerosis and the locus is a neuronal inclusion comprising TAR DNA-binding protein 43 (TDP-43), fused in sarcoma/translocated in liposarcoma (FUS/TLS), and superoxide dismutase-1 (SOD1). In some embodiments, the neurological disease or disorder is Huntington's Disease and the locus is neuronal intranuclear inclusions of Huntingtin. In some embodiments, the neurological disease or disorder is dementia with Lewy bodies and the locus is Lewy bodies comprising α-Synuclein, senile plaques comprising amyloid-O (Aβ), and neurofibrillary tangles comprising Tau protein. In some embodiments, the neurological disease or disorder is frontotemporal diseases and the locus is neuronal and glial inclusions composed of Tau, TDP-43, and FUS/TLS. In some embodiments, the neurological disease or disorder is multiple system atrophy, and the locus is glial cytoplasmic inclusions of α-synuclein. In some embodiments, the neurological disease or disorder is four-repeat tauopathy, and the locus is cytoplasmic inclusions predominantly composed of tau protein isoforms with four microtubule-binding domains.

The diseases that are associated with aggregation and/or accumulation of the protein (or another biomolecule) that exhibits abnormal production, aggregation, and/or deposition also include prion diseases, i.e., the transmissible spongiform encephalopathies such as bovine spongiform encephalopathy (BSE or mad cow disease) and Creutzfeldt-Jakob disease. These diseases feature senile plaques made of PrP protein. Accordingly, in some aspects, the present disclosure provides a method of treating a prion disease, the method comprising: (a) selecting the subject having a locus of deposition of PrP protein in the brain; (b) identifying a region of blood-brain barrier (BBB) for an ultrasound treatment, wherein the region is adjacent to or fully encompassing the locus; (c) administering a therapeutic agent to the subject; and (d) applying an ultrasound beam across the cranium of the subject to the region of BBB to facilitate transit of the therapeutic agent across BBB to the locus.

the present disclosure provides a method of treating a prion disease, the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from ¹⁸F-florbetapir, ¹⁸F-Florbetaben, ¹⁸F-Flortaucipir, ¹⁸F-Flutemetamol and ¹⁸F-Flourodopa (F-Dopa). In some embodiments, the SPECT reporter is selected from ^{99m}Tc-ECD (ethylcysteinate-dimer), ¹²³I-Ioflupane, and ^{99m}Tc-TRODAT-1.

### Alzheimer's Disease

Alzheimer's disease (AD) is a complex, progressively debilitating, and fatal neurodegenerative disease. AD is rapidly increasing in frequency as the world's population ages. There are currently an estimated 6.5 million individuals with AD in the US, and this number is expected to increase to more than 13 million by 2050. Approximately 15% of the US population over age 60 has prodromal AD and approximately 40% has preclinical AD. Similar trends are seen globally with an anticipated worldwide population of AD dementia patients exceeding 100 million by 2050 unless means of delaying, preventing, or treating AD are found. There is a significant need for therapeutics that halt or reverse the underlying pathology of AD.

Cellular and molecular mechanisms of AD are not well understood yet. Researchers have been reported that AD is associated with genetic and environmental factors and life-style. AD patients are heterogeneous in that they could be in preclinical AD continuum spanning up to two decades or more without exhibiting any clinical symptoms, i.e., mild cognitive impairment (MCI), AD dementia, or functional decline. Furthermore, misdiagnosis of AD patients is common in that 10-30% of individuals clinically diagnosed as AD dementia do not display AD neurodegeneration at autopsy.

Across all types of AD therapies, the failure rate is more than 99%, and for disease-modifying therapies (DMTs), the failure rate is 100%. Therefore, in addition to new approaches for developing therapeutic agents, approaches, such as those disclosed herein, for targeted delivery of the therapeutic agents is required.

Alzheimer's Disease is associated senile plaques composed of amyloid-β (Aβ), neurofibrillary tangles, which include Tau protein, neuronal inclusions of TDP-43 as well as Lewy bodies, which include α-Synuclein. Aβ is a relatively small peptide of 4 to 4.4 kDa that is the major component of amyloid deposits. Intracellular Aβ protein is widely found in neurons and it is associated with inflammatory and antioxidant activity, regulation of cholesterol transport, and activation of kinase enzyme. However, Aβ is one of the best known components in formation of neurodegenerative diseases including AD. Aβ is approximately composed of 36-43 amino acids and it originates from amyloid precursor protein (APP), which is a glycoprotein of 695-770 amino acids. APP can be cleaved into fragments by α, β, and γ secretases and Aβ protein is formed by the action of the β and γ secretases. Aβ protein contains two important regions which play a major role in the formation insoluble amyloid fibrils.

The microtubule associated Tau protein, the name of which is derived from “tubulin associated unit,” is highly expressed in brain. Microtubules are major proteins of the cytoskeleton. The main function of the Tau protein is to stabilize microtubules with binding to microtubules and to other proteins. To perform these functions, Tau protein is phosphorylated at normal level. Hyperphosphorylation of Tau protein is believed to cause conformational changes and aggregation of tau proteins. Other post-translational modifications such as glycosylation, glycation, polyamination, and nitration may play roles in aggregation. Accordingly, in some aspects, the present disclosure provides a method of treating Alzheimer's Disease (AD), wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating Alzheimer's Disease (AD), the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the protein that exhibits abnormal production, aggregation, and/or deposition is selected from amyloid-β peptide (Aβ), neurofibrillary tangles and tau protein. In some embodiments, the protein that exhibits abnormal production, aggregation, and/or deposition is selected from amyloid-O peptide (Aβ), TDP-43, α-synuclein and tau protein. In some embodiments, the locus is detected using an imaging technique that a reporter that is capable of binding to amyloid-β peptide (Aβ), neurofibrillary tangles and/or tau protein. In some embodiments, the imaging technique is PET. In some embodiments, the localizing comprises administering to the subject a PET reporter. In some embodiments, the PET reporter is selected from 18F-florbetapir, 18F-Florbetaben, 18F-Flortaucipir, and 18F-Flutemetamol. Additionally or alternatively, in some embodiments, the imaging technique is SPECT. In some embodiments, the localizing comprises administering to the subject a SPECT reporter. In some embodiments, the SPECT reporter is 99mTc-ECD (ethylcysteinate-dimer). In some embodiments, the therapeutic agent is selected from a nonspecific clearing antibody (e.g., intravenous immunoglobulin aka IVIg), an anti-amyloid-β antibody (e.g., aducanumab, gantenerumab, lecanemab, and donanemab), an anti-tau antibody (e.g., semorinemab, gosuranemab, tilavonemab, and zagotenemab), an anti-TREM2 antibody (e.g., AL002), donepezil, rivastigmine, memantine and galantamine and a combination thereof. In some embodiments, the therapeutic agent is aducanumab. Some therapeutic agents are disclosed in WO 2014/089500 and WO 2021/108861, and the content of which is incorporated herein by reference.

### Parkinson's Disease

Parkinson's disease (PD) is a long-term degenerative disorder of the central nervous system that causes unintended or uncontrollable movements, such as shaking, stiffness, and difficulty with balance and coordination. Symptoms usually begin gradually and worsen over time. As the disease progresses, people may have difficulty walking and talking. They may also have mental and behavioral changes, sleep problems, depression, memory difficulties, and fatigue. The occurrence of the illness is characterized by accumulation of misfolded α-synuclein protein in brain. Generally; anxiety, tremor, rigidity, depression, bradykinesia, and postural abnormalities are the most common symptoms in Parkinson's disease.

Lewy bodies (LBs), which mainly consist of α-syn, are neuropathological hallmarks of patients with Parkinson's disease (PD). It has been increasingly recognized, however, that PD is frequently associated with cognitive deficits, and that dementia eventually develops in a substantial number of patients.

α-synuclein is associated with a number of neurodegenerative diseases that are known as “Synucleinopathies.” Natively unfolded α-synuclein (α-Syn) is a 14 kDa and highly conserved protein that localize different regions of the brain. The name of protein was preferred as “α-synuclein” because of it shows synaptic and nuclear localization. α-Syn regulates dopamine neurotransmission by modulation of vesicular dopamine storage. It interacts with tubulin and can function like tau protein. Also, α-Syn shows a molecular chaperon activity in folding of SNARE (soluble N-ethylmaleimide-sensitive-factor attachment protein receptor) proteins. α-Syn plays crucial role in PD because α-Syn is a major fibrillary component for Lewy bodies. Two mutations, A53T and A30P, in the α-Syn gene and overexpression of wild type α-Syn are increases misfolding processes and aggregation. Also, accumulation of abnormal form of α-Syn can inhibit proteasomal functions. In PD brains, α-Syn is found to be phosphorylated at Ser87 and Ser129 in aggregates. These serine residues are phosphorylated with casein kinase 1 (CK1) and casein kinase 2 (CK2). It is believed that this post translational modification has a pathological role in fibrillation of α-Syn.

Accordingly, in some aspects, the present disclosure provides a method of treating Parkinson's Disease (PD), wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating Parkinson's Disease (PD), the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the protein that exhibits abnormal production, aggregation, and/or deposition is alpha-synuclein. In some embodiments, the locus is detected using an imaging technique that a reporter that is capable of binding to alpha-synuclein. In some embodiments, the imaging technique is PET. In some embodiments, the localizing comprises administering to the subject a PET reporter. In some embodiments, the PET reporter is 18F-Flourodopa (F-Dopa). Additionally or alternatively, in some embodiments, the imaging technique is SPECT. In some embodiments, the localizing comprises administering to the subject a SPECT reporter. In some embodiments, the SPECT reporter is 123I-Ioflupane, or 99mTc-TRODAT-1. In some embodiments, the therapeutic agent is selected from an anti-alpha-synuclein antibody (e.g., Cinpanemab, Prasinezumab, Lu AF82422, ABBV-0805, and MEDI1341), carbidopa-levodopa, selegiline, rasagiline, safinamide, entacapone, benztropine, tolcapone, opicapone, nuplazid, istradefylline and amantadine, and a combination thereof.

Multiple system atrophy features glial cytoplasmic inclusions of α-synuclein. Accordingly, in some aspects, the present disclosure provides a method of treating multiple system atrophy, wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating multiple system atrophy, the method comprising: the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the protein that exhibits abnormal production, aggregation, and/or deposition is alpha-synuclein. In some embodiments, the locus is detected using an imaging technique that a reporter that is capable of binding to alpha-synuclein. In some embodiments, the imaging technique is PET. In some embodiments, the localizing comprises administering to the subject a PET reporter. In some embodiments, the PET reporter is 18F-Flourodopa (F-Dopa). Additionally or alternatively, in some embodiments, the imaging technique is SPECT. In some embodiments, the localizing comprises administering to the subject a SPECT reporter. In some embodiments, the SPECT reporter is 123I-Ioflupane, or 99mTc-TRODAT-1. In some embodiments, the therapeutic agent is selected from an anti-alpha-synuclein antibody (e.g., Cinpanemab, Prasinezumab, Lu AF82422, ABBV-0805, and MEDI1341), carbidopa-levodopa, selegiline, rasagiline, safinamide, entacapone, benztropine, tolcapone, opicapone, nuplazid, istradefylline and amantadine, and a combination thereof.

Dementia with Lewy Bodies

Dementia with Lewy bodies features Lewy bodies, which include α-Synuclein, senile plaques that are mainly composed of amyloid-β (Aβ) and neurofibrillary tangles of Tau protein. Dementia with Lewy bodies (DLB) is a type of progressive dementia that leads to a decline in thinking, reasoning and independent function. Its features may include spontaneous changes in attention and alertness, recurrent visual hallucinations, REM sleep behavior disorder, and slow movement, tremors or rigidity. Mutations in genes known as SNCA and SNCB can cause dementia with Lewy bodies. Mutations in another gene called GBA or a certain version of a gene called APOE increase the risk of developing the condition, but are not a direct cause. Accordingly, in some aspects, the present disclosure provides a method of treating dementia with Lewy bodies, wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating dementia with Lewy bodies, the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the protein that exhibits abnormal production, aggregation, and/or deposition is alpha-synuclein. In some embodiments, the locus is detected using an imaging technique that a reporter that is capable of binding to alpha-synuclein and/or amyloid-O (Aβ). In some embodiments, the imaging technique is PET. In some embodiments, the localizing comprises administering to the subject a PET reporter. In some embodiments, the PET reporter is 18F-Flourodopa (F-Dopa), 18F-florbetapir, 18F-Florbetaben, 18F-Flortaucipir, and 18F-Flutemetamol. Additionally or alternatively, in some embodiments, the imaging technique is SPECT. In some embodiments, the localizing comprises administering to the subject a SPECT reporter. In some embodiments, the SPECT reporter is 123I-Ioflupane, or 99mTc-TRODAT-1. In some embodiments, the therapeutic agent is selected from an anti-alpha-synuclein antibody (e.g., Cinpanemab, Prasinezumab, Lu AF82422, ABBV-0805, and MEDI1341), rivastigmine, donepezil, galantamine, memantine, carbidopa-levodopa, and a combination thereof.

### Huntington's Disease

Huntington's disease (HD) is a genetic neurodegenerative disorder and the disease is caused by autosomal dominant inheritance. HD patients show involuntary muscle contractions, movement, and mental disorders. The disease is inherited as an autosomal dominant and effects brain and nervous systems. Huntington protein undergoes conformational changes with mutation and it shows aggregation tendency.

In HD, the neuropathology is characterized with accumulation of Htt protein aggregates. HD is caused by a number of CAG repeats in the gene. It is believed that the CAG repeats (polyQ) are the most important promoter for toxicity of Htt protein aggregates. The polyQ region starts at residue 18 and the number of glutamine residues are the most important marker in HD. Surprisingly, 40 or more CAG repeats are always generated neuropathy, while 35 or fewer CAG repeats are never generated neuropathy. However, in childhood, CAG repeats from 27 to 35 can develop neuropathy. Accordingly, in some aspects, the present disclosure provides a method of treating Huntington's Disease (HD), wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating Huntington's Disease (HD), the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the protein that exhibits abnormal production, aggregation, and/or deposition is huntingtin. In some embodiments, the locus is detected using an imaging technique that a reporter that is capable of binding to mutant Huntingtin or polyglutamine or metabolic changes associated with Huntington's Disease. In some embodiments, the imaging technique is PET. In some embodiments, the localizing comprises administering to the subject a PET reporter. In some embodiments, the PET reporter is selected from 11C-CHDI-180R, 11C-CHDI-626, 18F-CPFPX, 11C-KF18446, 18F-MK-9470, 11C-SCH-23390, 11C-NNC-112, 11C-Raclopride, 11C-Flumazenil, 11C-ABP-688, 11C-PK11195, 18F-PBRO6, 18F-JNJ42249152, 18F-MNI-659, 11C-IMA-107, and 11C-UCB-J. See Cybulska et al., Huntington's Disease: A Review of the Known PET Imaging Biomarkers and Targeting Radiotracers, Molecules 2020; 25(3), 482. Additionally or alternatively, in some embodiments, the imaging technique is SPECT. In some embodiments, the localizing comprises administering to the subject a SPECT reporter. In some embodiments, the SPECT reporter is ^{99m}Tc-HMPAO. In some embodiments, the therapeutic agent is selected from anti-huntingtin antibody and an anti-SEMA4D antibody (e.g., Pepinemab).

### Amyotrophic Lateral Sclerosis (ALS)

Amyotrophic lateral sclerosis (ALS) is a fatal neurodegenerative disorder characterized by degeneration of both upper and lower motor neurons, leading to progressive paralysis in muscles of the limbs, speech, swallowing and respiration. Patients suffering from amyotrophic lateral sclerosis have neuronal inclusions that include TAR DNA-binding protein 43 (TDP-43), fused in sarcoma/translocated in liposarcoma (FUS/TLS), and superoxide dismutase-1 (SOD1). ALS pathology is believed to begin at a single focal or multifocal sites and spread through the neuroaxis in a spatiotemporal manner. Insoluble TDP-43 from diseased brains has been reported to induce TDP-43 pathology in neuroblastoma cells that overexpress wtTDP-43 as detected by TDP-43 hyperphosphorylation, ubiquitination and aggregation. Accordingly, in some aspects, the present disclosure provides a method of treating amyotrophic lateral sclerosis (ALS), wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating amyotrophic lateral sclerosis (ALS), the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the protein that exhibits abnormal production, aggregation, and/or deposition is alpha-synuclein. In some embodiments, the locus is detected using an imaging technique that a reporter that is capable of binding to TAR DNA-binding protein 43 (TDP-43), fused in sarcoma/translocated in liposarcoma (FUS/TLS), and superoxide dismutase-1 (SOD1). In some embodiments, the imaging technique is PET. In some embodiments, the localizing comprises administering to the subject a PET reporter. In some embodiments, the PET reporter is 18F-fluorodeoxyglucose, 18F-florbetaben, 11C-flumazenil and/or 3H-APN-1607. Additionally or alternatively, in some embodiments, the imaging technique is SPECT. In some embodiments, the localizing comprises administering to the subject a SPECT reporter. In some embodiments, the SPECT reporter is ^{99m}Tc-ECD. In some embodiments, the therapeutic agent is selected from an anti-TDP-43 antibody, an anti-SOD1 antibody, riluzole, edaravone, and sodium phenylbutyrate and taurursodiol, or a combination thereof.

### Spinocerebellar Ataxia

Spinocerebellar ataxia (SCAs) is a complex group of neurodegenerative disorders characterized by progressive cerebellar ataxia of gait and limbs variably associated with ophthalmoplegia, pyramidal and extrapyramidal signs, dementia, pigmentary retinopathy and peripheral neuropathy. Disease onset is usually between 30 and 50 years of age, although early onset in childhood and onset in later decades after 60 years have been reported. The prognosis is variable depending on the underlying cause of the spinocerebellar ataxia subtype. Mutations in ATXN1, ATXN2, ATXN3, SCA4, SPTBN2, CACNAIA, ATXN7, KLHL1AS, ATXN10, SCA11, PPP2R2B, KCNC3, PRKCG, etc. are found in SCAs. In addition, seven spinocerebellar ataxia subtypes including SCAs 1, 2, 3/Machado-Joseph disease, 6, 7, 17 and dentatorubral pallidoluysian atrophy (DRPLA) are caused by the expansion of a CAG-repeat sequence in specific genes, leading to abnormally long polyQ tracts in the encoded proteins. Proteins with expanded stretches of polyglutamine appear to take on an abnormal configuration resulting in the formation and deposition of polyglutamine aggregates in disease neurons forming characteristic nuclear or cytoplasmic inclusions, which are neuropathological hallmarks in these diseases. Accordingly, in some aspects, the present disclosure provides a method of treating spinocerebellar ataxia, wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating spinocerebellar ataxia, the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the protein that exhibits abnormal production, aggregation, and/or deposition is a mutant protein having expanded polyglutamine tracts. In some embodiments, the locus is detected using an imaging technique that a reporter that is capable of binding to poly Q tracts or indicators of defective metabolism associated with spinocerebellar ataxia. In some embodiments, the imaging technique is PET. In some embodiments, the localizing comprises administering to the subject a PET reporter. In some embodiments, the PET reporter is ¹⁸F-fluorodeoxyglucose and/or ¹⁸F-SDM-8. Additionally or alternatively, in some embodiments, the imaging technique is SPECT. In some embodiments, the localizing comprises administering to the subject a SPECT reporter. In some embodiments, the SPECT reporter is ^{99m}Tc-TRODAT-1. In some embodiments, the therapeutic agent is selected from an anti-polyglutamine antibody.

### Frontotemporal Diseases

The clinical syndromes of frontotemporal dementia are clinically and neuropathologically heterogeneous, but processes such as neuroinflammation may be common across the disease spectrum. In recent years, attention has focused on understanding the pathogenic role of protein misfolding and aggregation, which is a cardinal feature of the post-mortem diagnostic criteria for frontotemporal lobar degeneration (FTLD). These diseases are associated neuronal and glial inclusions composed of Tau, TDP-43, and FUS/TLS. Frontotemporal dementia with parkinsonism-17 (FTDP-17) is a progressive neurodegenerative disease which is caused by mutations in the tau gene. The tau gene is mutated in familial FTDP-17 and this mutation accelerates formation of neurofibrillary tangles (NFTs) in the brain. Furthermore, hyperphosphorylation is promoted by this mutation. In some aspects, the present disclosure provides a method of treating frontotemporal dementia, the method comprising: wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating frontotemporal dementia, the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the locus is neuronal and glial inclusions composed of Tau, TDP-43, and FUS/TLS. In some embodiments, the protein that exhibits abnormal production, aggregation, and/or deposition is Tau, TDP-43, and FUS/TLS. In some embodiments, the locus is detected using an imaging technique that a reporter that is capable of binding to Tau, TDP-43, and FUS/TLS. In some embodiments, the imaging technique is PET. In some embodiments, the localizing comprises administering to the subject a PET reporter. In some embodiments, the PET reporter is selected from 18F-PI-2620, 18F-fluorodeoxyglucose and/or 18F-GE-180. Additionally or alternatively, in some embodiments, the imaging technique is SPECT. In some embodiments, the localizing comprises administering to the subject a SPECT reporter. In some embodiments, the SPECT reporter is ^{99m}Tc-HMPAO. In some embodiments, the therapeutic agent is an anti-tau antibody.

### Four-Repeat Tauopathy

Four-repeat (4R-) tauopathies are a group of neurodegenerative diseases defined by cytoplasmic inclusions of tau protein isoforms. Progressive supranuclear palsy, corticobasal degeneration, argyrophilic grain disease or glial globular tauopathy belong to the group of 4R-tauopathies. Tau is a microtubule-associated protein with versatile functions in the dynamic assembly of the neuronal cytoskeleton, and in these diseases, cytoplasmic inclusions predominantly composed of tau protein isoforms with four microtubule-binding domains are found. Moreover, Tau protein is generally located in axons, but in tauopathy, it is located in dendrites. Thus, neuron's transport system may be disintegrated and microtubule cannot function correctly. Accordingly, in some aspects, the present disclosure provides a method of treating a four-repeat (4R-) tauopathy, wherein a therapeutic agent and/or a microbubble composition will be, is being or has been administrated to the subject, the method comprising the steps of: sonicating a target volume to cause disruption of a target tissue therein and thereby increase a permeability thereof, wherein the target volume encompasses the locus and adjacent blood-brain barrier (BBB); and monitoring a degree of permeability of the target tissue caused by the disruption; and ceasing sonication of the target volume when the degree of permeability reaches a threshold, and thereby increasing delivery of a level of delivery of the therapeutic agent to the locus compared to a control. In some embodiments, the control is level of delivery of the therapeutic agent in the subject that has not received the sequence of acoustic pulses and/or the microbubble composition. In other embodiments, the control is level of delivery of the therapeutic agent in the subject prior to the administration of the sequence of acoustic pulses and/or the microbubble composition.

In some aspects, the present disclosure provides a method of treating a four-repeat (4R-) tauopathy, the method providing a predetermined range local dose of a therapeutic agent, the method comprising the steps of: (i) sonicating a target volume of to cause disruption of a target tissue therein and thereby increase a permeability thereof, wherein the target volume encompasses the locus and adjacent blood-brain barrier (BBB), wherein the subject that has received, will receive, is receiving a dose of the therapeutic agent, a microbubble composition and/or a detection marker, optionally on the same day; (ii) measuring the level of the detection marker in the target tissue, and thereby monitoring a local dose of the therapeutic agent at the locus caused by the disruption; (iii) ceasing sonication of the target volume when the local dose of the therapeutic agent reaches the predetermined range. In some embodiments, detection marker is: conjugated with a polymer (e.g., a dextran or a polyethylene glycol) that is in a defined molecular weight range corresponding to the molecular weight range of a therapeutic agent, or complexed a liposome having a defined size range corresponding to the size range of a therapeutic agent. In some embodiments, the detection marker is selected from a magnetic resonance imaging (MRI)-visible contrast agent, a positron emission tomography (PET) reporter and a single photon emission computed tomography (SPECT) reporter. In some embodiments, the MRI-visible contrast agent comprises ionic Gd. In some embodiments, the PET reporter is selected from 18F-florbetapir, 18F-Florbetaben, 18F-Flortaucipir, 18F-Flutemetamol and 18F-Flourodopa (F-Dopa). In some embodiments, the SPECT reporter is selected from 99mTc-ECD (ethylcysteinate-dimer), 123I-Ioflupane, and 99mTc-TRODAT-1.

In some embodiments, the protein that exhibits abnormal production, aggregation, and/or deposition is Tau protein. In some embodiments, the locus is detected using an imaging technique that a reporter that is capable of binding to Tau protein. In some embodiments, the imaging technique is PET. In some embodiments, the localizing comprises administering to the subject a PET reporter. In some embodiments, the PET reporter is selected from ¹⁸F-PI-2620, ¹⁸F-fluorodeoxyglucose and/or ¹⁸F-GE-180. Additionally or alternatively, in some embodiments, the imaging technique is SPECT. In some embodiments, the localizing comprises administering to the subject a SPECT reporter. In some embodiments, the SPECT reporter is ^{99m}Tc-HMPAO. In some embodiments, the therapeutic agent is an anti-tau antibody.

The disclosure will be further described in the following example, which does not limit the scope of the invention described in the claims.

### EXAMPLE

### Example 1. In Vivo Rat Study to Quantify and Predict the Acoustic and MRI Imaging Correlation to Delivery of Therapeutic Molecules Following Blood-Brain Barrier Disruption Caused by ExAblate 4000 Type 2 System, a Magnetic Resonance Image-Guided Focused Ultrasound (MRgFUS) System

This example has a goal of, inter alia, collecting data to inform correlation between ΔR2* maps to ΔR1 concentration maps and acoustic maps when injecting liposomes or iron oxide (or any other surrogate) along with DNA or monoclonal antibodies (mAb).

Male rats (Sprague-Dawley breed), 6 to 8 weeks of age and weighing between 150-300 g, are used as test subjects.

Molecules of various types and molecular sizes are transferred across the BBB following a disruption caused by ExAblate 4000 Type 2 System, using microbubbles. The sonication patterns are varied to achieve delivery. Various real time feedback/control mechanisms are implemented during such treatments; real time cavitation data is used to vary sonication power distribution within the treated volume as the treatment progresses. Various methods are used to evaluate the crossing of the molecules across the blood-brain barrier (liposomes or iron oxide (or other surrogates, MRI based quantification) along with DNA (qPCR) or mAb (ELISA) injection).

One or more of the following is monitored and evaluated:

Cavitation acoustic spectra. Cavitation is measured using hydrophones, either integral part of the system transducer or external ones.

Abnormalities in T2 and T2* MR images. Multi-planar, multi-sequence imaging of the brain is performed prior to and post treatment and during follow up procedures.

Gd labeling of the testing liposomes molecules and post treatment Gd permeation through the BBB. Multi-planar, multi-sequence T1 weighted imaging of the brain is performed prior to and post treatment and during follow up procedures.

Histopathological determining of tissue damage at selected treated sites including Petechia, Neural damage/effects, Immune response, and Ischemia.

Histopathological evaluation of randomly selected non treated locations for any micro pathological damage.

The study will enroll 128 animals in total and have two phases: the first phase includes 20 rats to test iron oxide dose for the following stage. (Range of dosages tested: 10-100 umol per rat Iron oxide) and the second phase includes three groups, two subgroups in each group.

The first group will test the iron oxide dose found in the first phase, along with DNA injection. Two DNA concentrations are tested: 0.135 mg/rat and 0.270 mg/rat (18 rats in each concentration, total of 36 rats).

The second group will test the iron oxide dose found in the first phase, along with mAb injection. Two mAb concentrations are tested: 5 mg/kg and 15 mg/kg (18 rats in each concentration, total of 36 rats).

The third group will test liposome injection (250 g rat), along with mAb injection. Two mAb concentrations are tested: 5 mg/kg and 15 mg/kg (18 rats in each concentration, total of 36 rats).

In all three groups we will treat single or multiple targets by focused ultrasound together with ultrasound contrast agent, possibly with multiple repeats in the same brain volume. Hydrophones, built into the sonication system or external ones, are used to assess the ultrasound activity of the contrast agent post sonication. Decisions on the intensity and number of further sonication are based on these measurements.

Following the procedure, the animals will undergo post-MRI imaging in order to assess the treatment success. In particular, T2 weighted, and T2* weighted images are used to assess tissue damage and T2* weighted and Gd enhanced T1 weighted imaging will provide information on BBB opening.

Selected animals may undergo additional examinations. Such examinations may include tests of the recovery of the BBB over time and treatment induced petechia or edema. Follow ups may take place up to 4 weeks post treatment. A maximal diversion of 48 hours is allowed. Both brain lobes are harvested and, in some cases, sent to pathology without disclosing the sonication parameters.

In selected animals both brain lobes are harvested, and selected ones are sent to pathology examination without disclosing the sonication parameters.

This study is expected to demonstrate one or more of: BBB permeability of liposome Gd and iron oxide is correlative to the permeability of the therapeutic molecules (DNA and mAb); T2* signal and acoustic maps are correlated with BBB permeability of the therapeutic molecules (DNA and mAb); T2 and T2* imaging does not indicate unintentional tissue damage and histopathology does not reveal unintentional microscopic tissue damage.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention. As used herein, all headings are simply for organization and are not intended to limit the disclosure in any manner. The content of any individual section may be equally applicable to all sections.

1. A system for disrupting target tissue for treatment, the system comprising:
an ultrasound transducer for sonicating a target volume to cause disruption of a target tissue therein and thereby increase a permeability thereof; and
a controller configured to:
(i) monitor a degree of permeability of the target tissue caused by the disruption; and
(ii) at least one of: (1) cause the ultrasound transducer to cease sonicating the target volume when the degree of permeability reaches a threshold; (2) present the degree of permeability of the target tissue to an operator; and (3) change an ultrasound intensity.

2. The system of claim 1, wherein the degree of permeability corresponds to an upper limit of a size distribution of molecules or molecular complexes that can pass through the disrupted target tissue.

3. The system of claim 2, wherein the degree of permeability is calculated based at least in part on:
(i) a measurement of a molecule that is present in the target tissue but substantially not present in tissues outside the target tissue under normal conditions, and/or
(ii) a measurement inside the target tissue of a detection marker that is administered a subject in which the degree of permeability is calculated.

4. The system of claim 3, wherein the target tissue is brain.

5. The system of claim 4, wherein the molecule that is predominantly present in the target tissue but substantially not present in tissues outside the target tissue under normal conditions is a brain protein.

6. The system of claim 3, wherein the detection marker and/or the is complexed with a polymer, a liposome, a quantum dot, or a dextran.

7. The system of claim 3 or claim 6, wherein the detection marker is selected from a magnetic resonance imaging (MRI)-visible contrast agent (e.g., ionic Gd), a magnetic resonance imaging (MRI)-visible contrast agent, a positron emission tomography (PET) reporter (e.g., ¹⁸F-florbetapir, ¹⁸F-Florbetaben, ¹⁸F-Flortaucipir, ¹⁸F-Flutemetamol and ¹⁸F-Flourodopa (F-Dopa) and a single photon emission computed tomography (SPECT) reporter (e.g., ^{99m}Tc-ECD (ethylcysteinate-dimer), ¹²³I-Ioflupane, and ^{99m}Tc-TRODAT-1).

8. The system of claim 1, wherein the degree of permeability corresponds to a concentration of molecules or molecular complexes having a size range and which can potentially pass through the disrupted target tissue.

9. The system of claim 8, wherein the degree of permeability is calculated based on:
(i) the measurement of a molecule that is in the size range, and present in the target tissue but not in tissues outside the target tissue, and/or
(ii) the measurement inside the target tissue of a detection marker that is in the size range and administered a subject in which the degree of permeability is calculated.

10. The system of any one of claims 3-9, wherein the degree of permeability is compared among at least two applications of sonication on different days administered to the same subject and/or among at least two applications of sonication administered to different subjects.

11. The system of claim 10, wherein the degree of permeability is normalized using the measurement of the detection agent or the molecule that is predominantly present in the target tissue but substantially not present in tissues outside the target tissue under normal conditions.

12. The system of claim 11, wherein the target tissue is brain, and the molecule that is predominantly present in the target tissue but substantially not present in tissues outside the target tissue under normal conditions is a brain protein.

13. The system of claim 12, wherein the species of NSE or S100B that is detected is homodimeric having molecular weights of about 78 kDa and 22 kDa, respectively.

14. The system of claim 12, wherein the measurement of the species of NSE and/or S100B is used for normalization of transit of a therapeutic agent in the molecular weight range of about 60 to about 100 kDa, and/or about 15 to about 30 kDa, respectively.

15. The system of claim 11, wherein the detection marker is:
conjugated with a polymer that is in a defined molecular weight range corresponding to the molecular weight range of a therapeutic agent, or
complexed a liposome having a defined size range corresponding to the size range of a therapeutic agent.

16. The system of claim 1, further comprising a MRI device.

17. The system of claim 16, wherein the controller is responsive to the MRI device and is further configured to evaluate the degree of permeability based on a measurement of at least one of ΔR₁, ΔR₂*, T₁ or T₂*.

18. The system of claim 17, wherein a relationship between the measurement and the degree of permeability is established by calibration.

19. The system of claim 18, wherein the calibration includes a machine learning component.

20. The system of claim 19, wherein the controller is configured to evaluate the degree of permeability without a contrast agent.

21. The system of claim 8, wherein the molecule or molecular complex includes a visualization agent.

22. The system of claim 21, wherein the visualization agent is a contrast agent.

23. The system of claim 22, wherein the contrast agent is ionic Gd (or iron oxide, or phosphate or sulfate derivatives).

24. The system of claim 21, wherein the visualization agent is a fluorophore.

25. The system of claim 8, wherein the size range corresponds to a monoclonal antibody, a viral vector, a liposome, a nucleic acid or a protein.

26. The system of claim 8, wherein the molecule or molecular complex is a liposome.

27. The system of claim 8, wherein the molecule or molecular complex is a quantum dot.

28. The system of claim 8, wherein the molecule or molecular complex is a dextran.

29. The system of claim 8, wherein the molecule or molecular complex is a protein.

30. The system of claim 8, wherein the molecule or molecular complex is a viral vector.

31. The system of claim 8, wherein the molecule or molecular complex is a nucleic acid.

32. The system of claim 1, wherein the transducer is configured also to detect acoustic signals.

33. The system of claim 32, wherein the controller is responsive to acoustic signals detected by the transducer and is further configured to evaluate the degree of permeability based on an acoustic measurement of a target species.

34. The system of claim 33, wherein the target species corresponds to a molecule or molecular complex coupled to an acoustic agent.

35. The system of claim 33, wherein the controller is further configured to evaluate the degree of permeability at least in part based on a combination of an acoustic measurement and an MRI image.

36. The system of claim 33, wherein the controller is further configured to update a target acoustic dose level at least in part based on a combination of an acoustic measurement and an MRI image.

37. The system of claim 36, wherein the controller is further configured to control an ultrasound treatment based at least in part on the updated target acoustic dose level.

38. The system of claim 37, wherein the updated target acoustic dose level is used repetitively during treatment.

39. The system of claim 34, wherein the degree of permeability corresponds to a concentration of agent-coupled molecules or molecular complexes having a size range and which have passed through the disrupted target tissue.

40. The system of claim 39, wherein the visualization agent is a contrast agent and the concentration is determined at least in part by either a wash-in or a wash-out time.

41. The system of claim 40, wherein the wash-out time corresponds to either raise or a decline in an acoustic signal characteristic with respect to a threshold.

42. The system of claim 29, wherein the acoustic agent is a contrast agent and the acoustic signal characteristic is reflected amplitude of an acoustic signal emitted by the transducer.

43. The system of any one of claims 1-42, further comprising an administration device for introducing microbubbles into the target tissue.

44. A method of disrupting target tissue for treatment, the method comprising the steps of:
sonicating a target volume to cause disruption of a target tissue therein and thereby increase a permeability thereof;
monitoring a degree of permeability of the target tissue caused by the disruption; and
ceasing sonication of the target volume when the degree of permeability reaches a threshold.

45. The method of claim 44, wherein the degree of permeability is calculated based at least in part on:
(i) a measurement of a level of a molecule that is present in the target tissue but substantially not present in tissues outside the target tissue under normal conditions in a biological sample from the subject in which the degree of permeability is being monitored, and/or
(ii) a measurement of a detection marker inside the target tissue, wherein the detection marker has been administered to a subject in which the degree of permeability is being monitored.

46. The method of claim 45, wherein the target tissue is brain and/or the biological sample is blood.

47. The method of claim 46, wherein the molecule that is present in the target tissue but substantially not present in tissues outside the target tissue under normal conditions is a brain protein.

48. The method of claim 47, wherein the species of NSE or S100B that is detected is homodimeric having molecular weights of about 78 kDa and 22 kDa, respectively.

49. The method of claim 48, wherein the measurement of the species of NSE and/or S100B is used for normalization of transit of a therapeutic agent in the molecular weight range of about 60 to about 100 kDa, and/or about 15 to about 30 kDa, respectively.

50. The method of claim 45, wherein the detection marker is:
conjugated with a polymer (e.g., a dextran or a polyethylene glycol) that is in a defined molecular weight range corresponding to the molecular weight range of a therapeutic agent, or
complexed a liposome having a defined size range corresponding to the size range of a therapeutic agent.

51. The method of claim 50, wherein the detection marker is selected from a magnetic resonance imaging (MRI)-visible contrast agent (e.g., ionic Gd), a magnetic resonance imaging (MRI)-visible contrast agent, a positron emission tomography (PET) reporter (e.g., ¹⁸F-florbetapir, ¹⁸F-Florbetaben, ¹⁸F-Flortaucipir, ¹⁸F-Flutemetamol and ¹⁸F-Flourodopa (F-Dopa) and a single photon emission computed tomography (SPECT) reporter (e.g., ^{99m}Tc-ECD (ethylcysteinate-dimer), ¹²³I-Ioflupane, and ^{99m}Tc-TRODAT-1).

52. The method of claim 44, wherein sonication comprises at least two applications on different days in the same patient or different applications in different patients.

53. The method of any one of claims 44-52, further comprising introducing microbubbles into the target tissue.

54. A method of comparing a degree of permeability of a target tissue in a subject among at least two applications of focused ultrasound treatment on different days, the method comprising:
(i) providing a biological sample from the subject that received a first application of sonication to a target volume to cause disruption of the target tissue therein and thereby increase a permeability thereof,
(ii) measuring the amount in the biological sample of a molecule that is present in the target tissue but substantially not present in tissues outside the target tissue under normal conditions,
(iii) monitoring a degree of permeability of the target tissue caused by the disruption;
(iv) providing a second biological sample from the subject that received a second application of sonication on a different day to a target volume to cause a second disruption of the target tissue therein and thereby increase a second permeability thereof,
(v) measuring in the second biological sample the amount of the molecule that is present in the target tissue but substantially not present in tissues outside the target tissue under normal conditions,
(vi) monitoring a degree of permeability of the target tissue caused by the second disruption; and
(vii) comparing a degree of permeability of the target tissue in a subject among at least two applications of sonication on different days.

55. The method of claim 54, wherein the target tissue is brain and/or the biological sample is blood.

56. The method of claim 54, wherein the molecule that is present in the target tissue but substantially not present in tissues outside the target tissue under normal conditions is a brain protein.

57. The method of claim 56, wherein the species of NSE or S100B that is detected is homodimeric having molecular weights of about 78 kDa and 22 kDa, respectively.

58. The method of claim 57, wherein the measurement of the species of NSE and/or S100B is used for normalization of transit of a therapeutic agent in the molecular weight range of about 60 to about 100 kDa, and/or about 15 to about 30 kDa, respectively.

59. The method of any one of claims 54-58, further comprising introducing microbubbles into the target tissue.

60. A method of comparing a degree of permeability of a target tissue in a subject among at least two applications of focused ultrasound treatment on different days, the method comprising:
(i) providing a subject that received a first application of sonication to a target volume to cause disruption of the target tissue therein and thereby increase a permeability thereof, wherein the subject has received a detection marker, optionally on the same day,
(ii) measuring the level of the detection marker in the target tissue, and thereby monitoring a degree of permeability of the target tissue caused by the disruption;
(iii) providing a second biological sample from the subject that received a second application of sonication on a different day to a target volume to cause a second disruption of the target tissue therein and thereby increase a second permeability thereof, optionally wherein the subject has received a detection marker on the same day,
(iv) measuring the level of the detection marker in the target tissue, and thereby monitoring a degree of permeability of the target tissue caused by the second disruption, and thereby monitoring a degree of permeability of the target tissue caused by the second disruption; and
(v) comparing a degree of permeability of the target tissue in a subject among at least two applications of sonication on different days.

61. The method of claim 60, wherein the target tissue is brain.

62. The method of claim 60, wherein detection marker is:
conjugated with a polymer (e.g., a dextran or a polyethylene glycol) that is in a defined molecular weight range corresponding to the molecular weight range of a therapeutic agent, or
complexed a liposome having a defined size range corresponding to the size range of a therapeutic agent.

63. The method of claim 62, wherein the detection marker is selected from a magnetic resonance imaging (MRI)-visible contrast agent, a magnetic resonance imaging (MRI)-visible contrast agent, a positron emission tomography (PET) reporter, and a single photon emission computed tomography (SPECT) reporter.

64. The method of claim 63, wherein the MRI-visible contrast agent comprises ionic Gd.

65. The method of claim 63, wherein the PET reporter is selected from ¹⁸F-florbetapir, ¹⁸F-Florbetaben, ¹⁸F-Flortaucipir, ¹⁸F-Flutemetamol and ¹⁸F-Flourodopa (F-Dopa).

66. The method of claim 63, wherein the SPECT reporter is selected from ^{99m}Tc-ECD (ethylcysteinate-dimer), ¹²³I-Ioflupane, and ^{99m}Tc-TRODAT-1.

67. The method of any one of claims 60-66, further comprising introducing microbubbles into the target tissue.

68. A method of treating a neurological disease or disorder in a subject in need thereof, wherein the neurological disease or disorder is characterized by having a locus of abnormal production, aggregation, and/or deposition of a protein or another biomolecule in the brain, wherein a therapeutic agent and/or a microbubble composition will be, is being or has been administrated to the subject, the method comprising the steps of:
sonicating a target volume to cause disruption of a target tissue therein and thereby increase a permeability thereof, wherein the target volume encompasses the locus and adjacent blood-brain barrier (BBB); and
monitoring a degree of permeability of the target tissue caused by the disruption; and
ceasing sonication of the target volume when the degree of permeability reaches a threshold, and thereby increasing delivery of a level of delivery of the therapeutic agent to the locus compared to a control.

69. The method of claim 68, wherein the control is level of delivery of the therapeutic agent in the subject that has not received the sequence of acoustic pulses and/or the microbubble composition.

70. The method of claim 68, wherein the control is level of delivery of the therapeutic agent in the subject prior to the administration of the sequence of acoustic pulses and/or the microbubble composition.

71. The method of any one of claims 68-70, wherein the neurological disease or disorder is selected from the Alzheimer's Disease (AD), Parkinson's Disease (PD), Huntington's Disease (HD), amyotrophic lateral sclerosis (ALS), dementia with Lewy bodies, spinocerebellar ataxia, and amyotrophic lateral sclerosis, frontotemporal diseases, multiple system atrophy, four-repeat tauopathy and prion diseases.

72. The method of any one of claims 68-71, wherein the locus is selected from senile plaques, neurofibrillary tangles, neuronal inclusions, Lewy bodies, glial inclusions, cytoplasmic inclusions, and polyglutamine aggregates.

73. The method of any one of claims 68-72, wherein the protein showing abnormal production, aggregation, and/or deposition is selected from amyloid-β (Aβ), Tau protein, of TDP-43, α-Synuclein, FUS/TLS, SOD1, and Huntingtin.

74. The method of any one of claims 68-73, wherein the therapeutic agent comprises a small molecule or a biologic drug.

75. The method of claim 74, wherein the therapeutic agent is or comprises a biologic drug.

76. The method of claim 75, wherein the therapeutic agent is selected from a gene therapy agent, an enzyme for enzyme replacement therapy, a vaccine, an antisense oligonucleotide (ASO), a protein therapeutic, a modified mRNA agent, and a RNAi agent.

77. The method of claim 75, wherein the therapeutic agent is or comprises an antibody, antibody-like molecule or an antigen-binding fragment thereof.

78. The method of claim 77, wherein the therapeutic agent specifically binds the protein or another biomolecule that that exhibits abnormal production, aggregation, and/or deposition.

79. The method of claim 77 or claim 78, wherein the therapeutic agent is selected from a nonspecific clearing antibody (e.g., intravenous immunoglobulin aka IVIg), an anti-amyloid-β antibody (e.g., aducanumab, gantenerumab, lecanemab, and donanemab), an anti-tau antibody (e.g., semorinemab, gosuranemab, tilavonemab, and zagotenemab), an anti-TREM2 antibody (e.g., AL002), an anti-alpha-synuclein antibody (e.g., Cinpanemab, Prasinezumab, Lu AF82422, ABBV-0805, and MEDI1331), and or a combination thereof.

80. The method of claim 74, wherein the therapeutic agent is or comprises a small molecule drug.

81. The method of claim 80, wherein the therapeutic agent provides one or more of synaptic plasticity, neuroprotection, reduction of inflammation, neurotransmitter receptor modulation, reduction of oxidative stress.

82. The method of claim 81, wherein the therapeutic agent is selected from donepezil, galantamine, rivastigmine, memantine, suvorexant, carbidopa-levodopa, selegiline, rasagiline, safinamide, entacapone, benztropine, tolcapone, opicapone, nuplazid, istradefylline and amantadine, and a combination thereof.

83. The method of any one of claims 68-82, wherein the therapeutic agent is formulated in a liposome.

84. The method of any one of claims 68-83, wherein the therapeutic agent is delivered via a viral vector.

85. The method of any one of claims 68-84, further comprising introducing microbubbles into the target tissue.

86. A method of treating a neurological disease or disorder in a subject in need thereof, wherein the neurological disease or disorder is characterized by having a locus of abnormal production, aggregation, and/or deposition of a protein or another biomolecule in the brain, the method providing a predetermined range local dose of a therapeutic agent, the method comprising the steps of:
sonicating a target volume to cause disruption of a target tissue therein and thereby increase a permeability thereof, wherein the target volume encompasses the locus and adjacent blood-brain barrier (BBB), wherein the subject has received, will receive, or is receiving: a dose of the therapeutic agent, a microbubble composition, and/or a detection marker, optionally on the same day;
measuring the level of the detection marker in the target tissue, and thereby monitoring a local dose of the therapeutic agent at the locus caused by the disruption;
ceasing sonication of the target volume when the local dose of the therapeutic agent reaches the predetermined range.

87. The method of claim 86, wherein detection marker is:
conjugated with a polymer (e.g., a dextran or a polyethylene glycol) that is in a defined molecular weight range corresponding to the molecular weight range of a therapeutic agent, or
complexed a liposome having a defined size range corresponding to the size range of a therapeutic agent.

88. The method of claim 86 or claim 87, wherein the detection marker is selected from a magnetic resonance imaging (MRI)-visible contrast agent, a positron emission tomography (PET) reporter and a single photon emission computed tomography (SPECT) reporter.

89. The method of claim 88, wherein the MRI-visible contrast agent comprises ionic Gd.

90. The method of claim 88, wherein the PET reporter is selected from ¹⁸F-florbetapir, ¹⁸F-Florbetaben, ¹⁸F-Flortaucipir, 18F-Flutemetamol and ¹⁸F-Flourodopa (F-Dopa).

91. The method of claim 88, wherein the SPECT reporter is selected from ^{99m}Tc-ECD (ethylcysteinate-dimer), ¹²³I-Ioflupane, and ^{99m}Tc-TRODAT-1.

92. The method of any one of claims 86-91, wherein the neurological disease or disorder is selected from the Alzheimer's Disease (AD), Parkinson's Disease (PD), Huntington's Disease (HD), amyotrophic lateral sclerosis (ALS), dementia with Lewy bodies, spinocerebellar ataxia, and amyotrophic lateral sclerosis, frontotemporal diseases, multiple system atrophy, four-repeat tauopathy and prion diseases.

93. The method of any one of claims 86-92, wherein the locus is selected from senile plaques, neurofibrillary tangles, neuronal inclusions, Lewy bodies, glial inclusions, cytoplasmic inclusions, and polyglutamine aggregates.

94. The method of any one of claims 86-93, wherein the protein showing abnormal production, aggregation, and/or deposition is selected from amyloid-β (Aβ), Tau protein, of TDP-43, α-Synuclein, FUS/TLS, SOD1, and Huntingtin.

95. The method of any one of claims 86-94, wherein the therapeutic agent comprises a small molecule or a biologic drug.

96. The method of claim 95, wherein the therapeutic agent is or comprises a biologic drug.

97. The method of claim 96, wherein the therapeutic agent is selected from a gene therapy agent, an enzyme for enzyme replacement therapy, a vaccine, an antisense oligonucleotide (ASO), a protein therapeutic, a modified mRNA agent, and a RNAi agent.

98. The method of claim 96, wherein the therapeutic agent is or comprises an antibody, antibody-like molecule or an antigen-binding fragment thereof.

99. The method of claim 98, wherein the therapeutic agent specifically binds the protein or another biomolecule that that exhibits abnormal production, aggregation, and/or deposition.

100. The method of claim 98 or claim 99, wherein the therapeutic agent is selected from a nonspecific clearing antibody (e.g., intravenous immunoglobulin aka IVIg), an anti-amyloid-β antibody (e.g., aducanumab, gantenerumab, lecanemab, and donanemab), an anti-tau antibody (e.g., semorinemab, gosuranemab, tilavonemab, and zagotenemab), an anti-TREM2 antibody (e.g., AL002), an anti-alpha-synuclein antibody (e.g., Cinpanemab, Prasinezumab, Lu AF82422, ABBV-0805, and MEDI1331), and or a combination thereof.

101. The method of claim 95, wherein the therapeutic agent is or comprises a small molecule drug.

102. The method of claim 101, wherein the therapeutic agent provides one or more of synaptic plasticity, neuroprotection, reduction of inflammation, neurotransmitter receptor modulation, reduction of oxidative stress.

103. The method of claim 102, wherein the therapeutic agent is selected from donepezil, galantamine, rivastigmine, memantine, suvorexant, carbidopa-levodopa, selegiline, rasagiline, safinamide, entacapone, benztropine, tolcapone, opicapone, nuplazid, istradefylline and amantadine, and a combination thereof.

104. The method of any one of claims 86-103, wherein the therapeutic agent is formulated in a liposome.

105. The method of any one of claims 86-104, wherein the therapeutic agent is delivered via a viral vector.

106. The method of any one of claims 86-105, further comprising introducing microbubbles into the target tissue.

107. A method of treating a central nervous system infection in a subject in need thereof, wherein a therapeutic agent will be, is being, or has been administrated to the subject, the method comprising the steps of:
sonicating a target volume to cause disruption of a target tissue therein and thereby increasing a permeability thereof;
monitoring a degree of permeability of the target tissue caused by the disruption; and
ceasing sonication of the target volume when the degree of permeability reaches a threshold.

108. The method of claim 107, wherein ceasing the sonication of the target volume increases delivery of a level of delivery of the therapeutic agent to a locus compared to a control.

109. The method of claim 107, wherein ceasing the sonication of the target volume includes ceasing the sonication when a local dose of the therapeutic agent reaches a predetermined range.

110. The method of any one of claims 107-109, wherein the therapeutic agent comprises at least one of an antibiotic, an anti-viral, an anti-retroviral, or an anti-fungal.

111. The method of any one of claims 107-110, further comprising introducing microbubbles into at the target volume.

112. A method of treating a congenital enzyme defect in a subject in need thereof, wherein a therapeutic agent will be, is being, or has been administrated to the subject, the method comprising the steps of:
sonicating a target volume to cause disruption of a target tissue therein and thereby increasing a permeability thereof;
monitoring a degree of permeability of the target tissue caused by the disruption; and
ceasing sonication of the target volume when the degree of permeability reaches a threshold.

113. The method of claim 112, wherein ceasing the sonication of the target volume increases delivery of a level of delivery of the therapeutic agent to a locus compared to a control.

114. The method of claim 112, wherein ceasing the sonication of the target volume includes ceasing the sonication when a local dose of the therapeutic agent reaches a predetermined range.

115. The method of any one of claims 112-114, wherein the therapeutic agent comprises an enzyme replacement therapy.

116. The method of any one of claims 112-115, further comprising introducing microbubbles into at the target volume.
